SEQUENCE LISTING (52 sequences)

SEQ ID NO: 1 - DNA, 25 bp, Artificial Sequence
  (CCT)8C inhibitory olionucleotide
cctcctcctcctcctcctcctcctc

SEQ ID NO: 2 - DNA, 26 bp, Artificial Sequence
  (CCT)8CC inhibitory oligonucleotide
cctcctcctcctcctcctcctcctcc

SEQ ID NO: 3 - DNA, 27 bp, Artificial Sequence
  (CCT)9 inhibitory oligonucleotide
cctcctcctcctcctcctcctcctcct

SEQ ID NO: 4 - DNA, 28 bp, Artificial Sequence
  Inhibitory oligonucleotide
cctcctcctcctcctcctcctcctcctc

SEQ ID NO: 5 - DNA, 29 bp, Artificial Sequence
  Inhibitory oligonucleotide
cctcctcctcctcctcctcctcctcctcc

SEQ ID NO: 6 - DNA, 30 bp, Artificial Sequence
  (CCT)10 inhibitory oligonucleotide
cctcctcctcctcctcctcctcctcctcct

SEQ ID NO: 7 - DNA, 31 bp, Artificial Sequence
  (CCT)10C inhibitory oligonucleotide
cctcctcctcctcctcctcctcctcctcctc

SEQ ID NO: 8 - DNA, 32 bp, Artificial Sequence
  (CCT)10CC inhibitory oligonucleotide
cctcctcctcctcctcctcctcctcctcctcc

SEQ ID NO: 9 - DNA, 33 bp, Artificial Sequence
  (CCT)11 inhibitory oligonucleotide
cctcctcctcctcctcctcctcctcctcctcct

SEQ ID NO: 10 - DNA, 34 bp, Artificial Sequence
  (CCT)11C inhibitory oligonucleotide
cctcctcctcctcctcctcctcctcctcctcctc

SEQ ID NO: 11 - DNA, 35 bp, Artificial Sequence
  (CCT)11CC inhibitory oligonucleotide
cctcctcctcctcctcctcctcctcctcctcctcc

SEQ ID NO: 12 - DNA, 36 bp, Artificial Sequence
  (CCT)12 inhibitory oligonucleotide
cctcctcctcctcctcctcctcctcctcctcctcct

SEQ ID NO: 13 - DNA, 42 bp, Artificial Sequence
  (CCT)14 inhibitory oligonucleotide
cctcctcctcctcctcctcctcctcctcctcctcctcctcct

SEQ ID NO: 14 - DNA, 48 bp, Artificial Sequence
  (CCT)16 inhibitory oligonucleotide
cctcctcctcctcctcctcctcctcctcctcctcctcctcctcctcct

SEQ ID NO: 15 - DNA, 18 bp, Artificial Sequence
  (CCT)6 inhibitory oligonucleotide
cctcctcctcctcctcct

SEQ ID NO: 16 - DNA, 21 bp, Artificial Sequence
  (CCT)7 inhibitory oligonucleotide
cctcctcctcctcctcctcct

SEQ ID NO: 17 - DNA, 22 bp, Artificial Sequence
  CpG2395 TLR9 stimulatory oligonucleotide
tcgtcgttttcggcgcgcgccg

SEQ ID NO: 18 - DNA, 20 bp, Artificial Sequence
  CpG1826 TLR9 stimulatory oligonucleotide
tccatgacgttcctgacgtt

SEQ ID NO: 19 - DNA, 20 bp, Artificial Sequence
  CpG2216 stimulatory oligonucleotide
gggggacgatcgtcgggggg

SEQ ID NO: 20 - DNA, 24 bp, Artificial Sequence
  (CCT)8 inhibitory oligonucleotide
cctcctcctcctcctcctcctcct

SEQ ID NO: 21 - DNA, 36 bp, Artificial Sequence; identical to SEQ ID NO: 12
  Inhibitory oligonucleotide
  modified_base (1)..(2): phosphorothioate-modified
  modified_base (4)..(36): phosphorothioate-modified

SEQ ID NO: 22 - DNA, 36 bp, Artificial Sequence; identical to SEQ ID NO: 12
  Inhibitory oligonucleotide
  modified_base (1)..(2): phosphorothioate modified
  modified_base (4)..(5): phosphorothioate modified
  modified_base (7)..(36): phosphorothioate modified

SEQ ID NO: 23 - DNA, 36 bp, Artificial Sequence; identical to SEQ ID NO: 12
  Inhibitory oligonucleotide
  modified_base (1)..(2): phosphorothioate-modified
  modified_base (4)..(5): phosphorothioate-modified
  modified_base (7)..(8): phosphorothioate-modified
  modified_base (10)..(36): phosphorothioate-modified

SEQ ID NO: 24 - DNA, 36 bp, Artificial Sequence; identical to SEQ ID NO: 12
  Inhibitory oligonucleotide
  modified_base (1)..(2): phosphorothioate-modified
  modified_base (4)..(5): phosphorothioate-modified
  modified_base (7)..(8): phosphorothioate-modified
  modified_base (10)..(11): phosphorothioate-modified
  modified_base (13)..(36): phosphorothioate-modified

SEQ ID NO: 25 - DNA, 36 bp, Artificial Sequence
  Inhibitory oligonucleotide
  modified_base (1)..(2): phosphorothioate-modified
  modified_base (4)..(5): phosphorothioate-modified
  modified_base (7)..(8): phosphorothioate-modified
  modified_base (10)..(11): phosphorothioate-modified
  modified_base (13)..(18): phosphorothioate-modified
  modified_base (20)..(36): phosphorothioate-modified
cctcctcctcctcccctctctcctcctcctcctcct

SEQ ID NO: 26 - DNA, 36 bp, Artificial Sequence; identical to SEQ ID NO: 12
  Inhibitory oligonucleotide
  modified_base (1)..(2): phosphorothioate-modified
  modified_base (4)..(5): phosphorothioate-modified
  modified_base (7)..(8): phosphorothioate-modified
  modified_base (10)..(11): phosphorothioate-modified
  modified_base (13)..(14): phosphorothioate-modified
  modified_base (16)..(17): phosphorothioate-modified
  modified_base (19)..(36): phosphorothioate-modified

SEQ ID NO: 27 - DNA, 36 bp, Artificial Sequence; identical to SEQ ID NO: 12
  Inhibitory oligonucleotide
  modified_base (1)..(2): phosphorothioate-modified
  modified_base (4)..(5): phosphorothioate-modified
  modified_base (7)..(8): phosphorothioate-modified
  modified_base (10)..(11): phosphorothioate-modified
  modified_base (13)..(14): phosphorothioate-modified
  modified_base (16)..(17): phosphorothioate-modified
  modified_base (19)..(20): phosphorothioate-modified
  modified_base (22)..(36): phosphorothioate-modified

SEQ ID NO: 28 - DNA, 36 bp, Artificial Sequence; identical to SEQ ID NO: 12
  Inhibitory oligonucleotide
  modified_base (1)..(2): phosphorothioate-modified
  modified_base (4)..(5): phosphorothioate-modified
  modified_base (7)..(8): phosphorothioate-modified
  modified_base (10)..(11): phosphorothioate-modified
  modified_base (13)..(14): phosphorothioate-modified
  modified_base (16)..(17): phosphorothioate-modified
  modified_base (19)..(20): phosphorothioate-modified
  modified_base (22)..(23): phosphorothioate-modified
  modified_base (25)..(36): phosphorothioate-modified

SEQ ID NO: 29 - DNA, 36 bp, Artificial Sequence; identical to SEQ ID NO: 12
  Inhibitory oligonucleotide
  modified_base (1)..(2): phosphorothioate-modified
  modified_base (4)..(5): phosphorothioate-modified
  modified_base (7)..(8): phosphorothioate-modified
  modified_base (10)..(11): phosphorothioate-modified
  modified_base (13)..(14): phosphorothioate-modified
  modified_base (16)..(17): phosphorothioate-modified
  modified_base (19)..(20): phosphorothioate-modified
  modified_base (22)..(23): phosphorothioate-modified
  modified_base (25)..(36): phosphorothioate-modified

SEQ ID NO: 30 - DNA, 36 bp, Artificial Sequence; identical to SEQ ID NO: 12
  Inhibitory oligonucleotide
  modified_base (1)..(2): phosphorothioate-modified
  modified_base (4)..(5): phosphorothioate-modified
  modified_base (7)..(8): phosphorothioate-modified
  modified_base (10)..(11): phosphorothioate-modified
  modified_base (13)..(14): phosphorothioate-modified
  modified_base (16)..(17): phosphorothioate-modified
  modified_base (19)..(20): phosphorothioate-modified
  modified_base (22)..(23): phosphorothioate-modified
  modified_base (25)..(26): phosphorothioate-modified
  modified_base (28)..(36): phosphorothioate-modified

SEQ ID NO: 31 - DNA, 37 bp, Artificial Sequence
  Inhibitory oligonucleotide
  modified_base (1)..(2): phosphorothioate-modified
  modified_base (4)..(5): phosphorothioate-modified
  modified_base (7)..(8): phosphorothioate-modified
  modified_base (10)..(11): phosphorothioate-modified
  modified_base (13)..(14): phosphorothioate-modified
  modified_base (16)..(17): phosphorothioate-modified
  modified_base (19)..(20): phosphorothioate-modified
  modified_base (22)..(23): phosphorothioate-modified
  modified_base (25)..(26): phosphorothioate-modified
  modified_base (28)..(29): phosphorothioate-modified
  modified_base (31)..(37): phosphorothioate-modified
cctcctcctcctcctcctcctcctcctccttcctcct

SEQ ID NO: 32 - DNA, 36 bp, Artificial Sequence; identical to SEQ ID NO: 12
  Inhibitory oligonucleotide
  modified_base (1)..(2): phosphorothioate-modified
  modified_base (4)..(5): phosphorothioate-modified
  modified_base (7)..(8): phosphorothioate-modified
  modified_base (10)..(11): phosphorothioate-modified
  modified_base (13)..(14): phosphorothioate-modified
  modified_base (16)..(17): phosphorothioate-modified
  modified_base (19)..(20): phosphorothioate-modified
  modified_base (22)..(23): phosphorothioate-modified
  modified_base (25)..(26): phosphorothioate-modified
  modified_base (28)..(29): phosphorothioate-modified
  modified_base (31)..(32): phosphorothioate-modified
  modified_base (34)..(36): phosphorothioate-modified

SEQ ID NO: 33 - DNA, 36 bp, Artificial Sequence; identical to SEQ ID NO: 12
  Inhibitory oligonucleotide
  modified_base (1)..(2): phosphorothioate-modified
  modified_base (4)..(5): phosphorothioate-modified
  modified_base (7)..(8): phosphorothioate-modified
  modified_base (10)..(11): phosphorothioate-modified
  modified_base (13)..(14): phosphorothioate-modified
  modified_base (16)..(17): phosphorothioate-modified
  modified_base (19)..(20): phosphorothioate-modified
  modified_base (22)..(23): phosphorothioate-modified
  modified_base (25)..(26): phosphorothioate-modified
  modified_base (28)..(29): phosphorothioate-modified
  modified_base (31)..(32): phosphorothioate-modified
  modified_base (34)..(35): phosphorothioate-modified

SEQ ID NO: 34 - DNA, 36 bp, Artificial Sequence; identical to SEQ ID NO: 12
  Inhibitory oligonucleotide
  modified_base (1)..(2): phosphorothioate-modified
  modified_base (4)..(17): phosphorothioate-modified
  modified_base (19)..(36): phosphorothioate-modified

SEQ ID NO: 35 - DNA, 36 bp, Artificial Sequence; identical to SEQ ID NO: 12
  Inhibitory oligonucleotide
  modified_base (1)..(5): phosphorothioate-modified
  modified_base (6)..(23): phosphorothioate-modified
  modified_base (25)..(36): phosphorothioate-modified

SEQ ID NO: 36 - DNA, 36 bp, Artificial Sequence; identical to SEQ ID NO: 12
  Inhibitory oligonucleotide
  modified_base (1)..(8): phosphorothioate-modified
  modified_base (10)..(26): phosphorothioate-modified
  modified_base (28)..(36): phosphorothioate-modified

SEQ ID NO: 37 - DNA, 36 bp, Artificial Sequence; identical to SEQ ID NO: 12
  Inhibitory oligonucleotide
  modified_base (1)..(14): phosphorothioate-modified
  modified_base (16)..(32): phosphorothioate-modified
  modified_base (34)..(36): phosphorothioate-modified

SEQ ID NO: 38 - DNA, 36 bp, Artificial Sequence; identical to SEQ ID NO: 12
  Inhibitory oligonucleotide
  modified_base (1)..(2): phosphorothioate-modified
  modified_base (4)..(14): phosphorothioate-modified
  modified_base (16)..(26): phosphorothioate-modified
  modified_base (28)..(36): phosphorothioate-modified

SEQ ID NO: 39 - DNA, 36 bp, Artificial Sequence; identical to SEQ ID NO: 12
  Inhibitory oligonucleotide
  modified_base (1)..(5): phosphorothioate-modified
  modified_base (7)..(17): phosphorothioate-modified
  modified_base (19)..(29): phosphorothioate-modified
  modified_base (31)..(36): phosphorothioate-modified

SEQ ID NO: 40 - DNA, 36 bp, Artificial Sequence; identical to SEQ ID NO: 12
  Inhibitory oligonucleotide
  modified_base (1)..(8): phosphorothioate-modified
  modified_base (10)..(20): phosphorothioate-modified
  modified_base (22)..(32): phosphorothioate-modified
  modified_base (34)..(36): phosphorothioate-modified

SEQ ID NO: 41 - DNA, 36 bp, Artificial Sequence; identical to SEQ ID NO: 12
  Inhibitory oligonucleotide
  modified_base (1)..(11): phosphorothioate-modified
  modified_base (13)..(23): phosphorothioate-modified
  modified_base (25)..(35): phosphorothioate-modified

SEQ ID NO: 42 - DNA, 36 bp, Artificial Sequence; identical to SEQ ID NO: 12
  Inhibitory olligonucleotide
  modified_base (1)..(2): phosphorothioate-modified
  modified_base (4)..(11): phosphorothioate-modified
  modified_base (13)..(20): phosphorothioate-modified
  modified_base (22)..(29): phosphorothioate-modified
  modified_base (31)..(36): phosphorothioate-modified

SEQ ID NO: 43 - DNA, 36 bp, Artificial Sequence; identical to SEQ ID NO: 12
  Inhibitory oligonculeotide
  modified_base (1)..(5): phosphorothioate-modified
  modified_base (7)..(14): phosphorothioate-modified
  modified_base (16)..(23): phosphorothioate-modified
  modified_base (25)..(32): phosphorothioate-modified
  modified_base (34)..(36): phosphorothioate-modified

SEQ ID NO: 44 - DNA, 36 bp, Artificial Sequence; identical to SEQ ID NO: 12
  Inhibitory oligonucleotide
  modified_base (1)..(8): phosphorothioate-modified
  modified_base (10)..(17): phosphorothioate-modified
  modified_base (19)..(26): phosphorothioate-modified
  modified_base (28)..(35): phosphorothioate-modified

SEQ ID NO: 45 - DNA, 36 bp, Artificial Sequence; identical to SEQ ID NO: 12
  Inhibitory oligonucleotide
  modified_base (1)..(2): phosphorothioate-modified
  modified_base (4)..(8): phosphorothioate-modified
  modified_base (10)..(14): phosphorothioate-modified
  modified_base (16)..(20): phosphorothioate-modified
  modified_base (22)..(26): phosphorothioate-modified
  modified_base (28)..(36): phosphorothioate-modified

SEQ ID NO: 46 - DNA, 36 bp, Artificial Sequence; identical to SEQ ID NO: 12
  Inhibitory oligonucleotide
  modified_base (1)..(8): phosphorothioate-modified
  modified_base (10)..(14): phosphorothioate-modified
  modified_base (16)..(20): phosphorothioate-modified
  modified_base (22)..(26): phosphorothioate-modified
  modified_base (28)..(29): phosphorothioate-modified
  modified_base (31)..(36): phosphorothioate-modified

SEQ ID NO: 47 - DNA, 36 bp, Artificial Sequence; identical to SEQ ID NO: 12
  Inhibitory oligonucleotide
  modified_base (1)..(14): phosphorothioate-modified
  modified_base (16)..(20): phosphorothioate-modified
  modified_base (22)..(26): phosphorothioate-modified
  modified_base (28)..(29): phosphorothioate-modified
  modified_base (31)..(32): phosphorothioate-modified
  modified_base (34)..(36): phosphorothioate-modified

SEQ ID NO: 48 - DNA, 36 bp, Artificial Sequence; identical to SEQ ID NO: 12
  Inhibitory oligonucleotide
  modified_base (1)..(20): phosphorothioate-modified
  modified_base (22)..(26): phosphorothioate-modified
  modified_base (28)..(29): phosphorothioate-modified
  modified_base (31)..(32): phosphorothioate-modified
  modified_base (34)..(35): phosphorothioate-modified

SEQ ID NO: 49 - DNA, 36 bp, Artificial Sequence; identical to SEQ ID NO: 12
  Inhibitory oligonucleotide
  modified_base (1)..(2): phosphorothioate-modified
  modified_base (4)..(8): phosphorothioate-modified
  modified_base (10)..(14): phosphorothioate-modified
  modified_base (16)..(20): phosphorothioate-modified
  modified_base (22)..(26): phosphorothioate-modified
  modified_base (28)..(32): phosphorothioate-modified
  modified_base (34)..(36): phosphorothioate-modified

SEQ ID NO: 50 - DNA, 36 bp, Artificial Sequence; identical to SEQ ID NO: 12
  Inhibitory oligonucleotide
  modified_base (1)..(5): phosphorothioate-modified
  modified_base (7)..(11): phosphorothioate-modified
  modified_base (13)..(17): phosphorothioate-modified
  modified_base (19)..(23): phosphorothioate-modified
  modified_base (25)..(29): phosphorothioate-modified
  modified_base (31)..(35): phosphorothioate-modified

SEQ ID NO: 51 - DNA, 20 bp, Artificial Sequence
  MYD 88 forward primer
gttgaagactgggcttgtcc

SEQ ID NO: 52 - DNA, 19 bp, Artificial Sequence
  MYD 88 reverse primer
aggaggcagggcagaagta

(19) United States
(12) Patent Application Publication
(10) Pub. No.: US 2017/0240896 A1
(43) Pub. Date: Aug. 24, 2017

(54) Inhibitory Oligonucleotides for Treating Tumors

(71) Applicant: SBI BIOTECH, CO., LTD., Tokyo (JP)

(72) Inventors: Dapeng Qian, Briarcliff Manor, NY (US); Eiji Esashi, Tokyo (JP)

(21) Appl. No.: 15/115,095

(22) PCT Filed: Feb. 6, 2015
(86) PCT No.: PCT/US2015/014902

§ 371 Date: Jul. 28, 2016

Related U.S. Application Data
(60) Provisional application No. 61/937,376, filed on Feb. 7, 2014.

(51) Int. Cl. C12N 15/117; A61K 45/06; A61K 31/713

(52) U.S. Cl. CPC C12N 15/117; A61K 31/713; A61K 45/06; C12N 2310/315; C12N 2320/31; C12N 2310/17

(57) ABSTRACT

A method for treating B-cell lymphoma in a subject that has been diagnosed as having a B-cell lymphoma characterized by a mutation in MYD88 and is in need of such treatment is presented. The lymphoma is treated with an oligonucleotide having a sequence 5′-(CCT)n-3′. The B-cell lymphoma may be activated B-cell-like (ABC) subtype of diffuse large B-cell lymphoma (DLBCL) or Waldenstrom's macroglobulinemia (WM).

### CROSS REFERENCE TO RELATED APPLICATIONS

This application claims the benefit of, and priority to, U.S. Provisional Patent Application Ser. No. 61/937,376, filed Feb. 7, 2014, the entire disclosures of which are hereby incorporated by reference in their entireties.

### FIELD OF THE INVENTION

The present invention relates to oligonucleotides and use thereof for treating tumors, such as B-cell lymphomas.

### BACKGROUND OF THE INVENTION

The immune system protects human body from bacterial, parasitic, fungal, viral infections and from the growth of tumor cells. Immunity can be classified as innate immunity or adaptive immunity. Innate immune responses typically occur immediately upon infection for providing an early barrier to infectious disease whereas adaptive immune responses occur later with the generation of antigen-specific long term protective immunity.

However, immune response can sometimes be unwanted and cause immune-mediated disorder, including autoimmune disease, graft rejection, hypersensitivity, diseases associated with the over-stimulation of host's immune system by microbes, and Toll-like receptor (TLR)-mediated diseases. Toll-like receptor (TLR)-mediated disease is a disorder caused by the activation of Toll like receptors (TLRs).

TLRs are a family of receptors that recognize microbe derived molecular structures (pathogen-associated molecular patterns or PAMPs). TLR expressing immune cells are activated upon binding of PAMPs. TLRs recognize a range of pathogen-derived products and activated. Lipopolysaccharide (LPS) of bacteria recognized by TLR4, lipotechoic acid and diacylated lipopeptides by TLR2-TLR6 dimmer, triacylated lipopeptides by TLR2-TLR1 dimmer, CpG containing oligonucleotide (CpG ODN) synthesized or derived from either viruses or bacteria by TLR9, bacterial flagellin by TLR5, zymosan by TLR2-TLR6 dimmer, F protein from respiratory syncytial virus (RSV) by TLR4, viral-derived double-stranded RNA (dsRNA) and poly I:C, a synthetic analog of dsRNA by TLR3; viral DNA by TLR9, single-stranded viral RNA (VSV and flu virus) and synthetic guanosine analogs such as imidazoquinolines and imiquimod by TLR7 and TLR8 (Foo Y. Liew, et al. Nature Reviews Immunology. Vol 5, June 2005, 446-458). The signaling mechanisms leading to the induction of type I IFNs differ depending on the TLR activated. They involve the interferon regulatory factors, IRFs, a family of transcription factors known to play a critical role in antiviral defense, cell growth and immune regulation. Three IRFs (IRF3, IRF5 and IRF7) function as direct transducers of virus-mediated TLR signaling. TLR3 and TLR4 activate IRF3 and IRF7 (Doyle S. et al. Immunity. 2002 17(3):251-63), while TLR7 and TLR8 activate IRF5 and IRF7 (Schoenemeyer A. et al. J Biol Chem. 2005 280(17):17005-12). Furthermore, type I IFN production stimulated by TLR9 ligand CpG-A has been shown to be mediated by Phosphoinositide 3-kinases (PI3K) and mTOR (Costa-Mattioli M et al. Nat Rev Drug Discov, 2010 9: 293-307).

### SUMMARY OF THE INVENTION

In one aspect, the present invention provides a method for treating B-cell lymphoma in a subject that has been diagnosed as having a B-cell lymphoma characterized by a mutation in MYD88 and is in need of such treatment, comprising: administering to the subject a pharmaceutical composition comprising a therapeutically effective amount of an oligonucleotide having a sequence of 5′-(CCT)ₙ-3′, wherein n is an integer from 2 to 50, and a pharmaceutically acceptable carrier.

In some embodiments, the oligonucleotide having a sequence of 5′-(CCT)nCm-3, n is an integer from 6 to 16, m is 0, 1, or 2.

In some embodiments, the B-cell lymphoma is selected from the group consisting of: Waldenstrom's macroglobulinemia (WM), activated B-cell-like (ABC) subtype of diffuse large B-cell lymphoma (DLBCL), and gastric mucosa-associated lymphoid tissue (MALT) lymphoma.

In some embodiments, the mutation in MYD88 comprises L265P, M232T, S243N, or T294P.

In some embodiments, wherein the oligonucleotide comprises a sequence selected from the group consisting of:

(SEQ ID NO: 1)
5′-cctcctcctcctcctcctcctcctc-3′,
(SEQ ID NO: 2)
5′-cctcctcctcctcctcctcctcctcc-3′,
(SEQ ID NO: 3)
5′-cctcctcctcctcctcctcctcctcct-3′,
(SEQ ID NO: 4)
5′-cctcctcctcctcctcctcctcctcctc-3′,
(SEQ ID NO: 5)
5′-cctcctcctcctcctcctcctcctcctcc-3′,
(SEQ ID NO: 6)
5′-cctcctcctcctcctcctcctcctcctcct-3′,
(SEQ ID NO: 7)
5′-cctcctcctcctcctcctcctcctcctcctc-3′,
(SEQ ID NO: 8)
5′-cctcctcctcctcctcctcctcctcctcctcc-3′,
(SEQ ID NO: 9)
5′-cctcctcctcctcctcctcctcctcctcctcct-3′,
(SEQ ID NO: 10)
5′-cctcctcctcctcctcctcctcctcctcctcctc-3′,
(SEQ ID NO: 11)
5′-cctcctcctcctcctcctcctcctcctcctcctcc-3′,
(SEQ ID NO: 12)
5′-cctcctcctcctcctcctcctcctcctcctcctcct-3′,
(SEQ ID NO: 13)
5′-cctcctcctcctcctcctcctcctcctcctcctcctcctcct-3′,
(SEQ ID NO: 14)
5′-cctcctcctcctcctcctcctcctcctcctcctcctcctcctcctcc
t-3′,
(SEQ ID NO: 15)
5′-cctcctcctcctcctcct-3′,
and
(SEQ ID NO: 16)
5′-cctcctcctcctcctcctcct-3′.

In some embodiments, phosphate backbone of the oligonucleotide is unmodified.

In some embodiments, phosphate backbone of the oligonucleotide is partially or completely phosphorothioate-modified.

In some embodiments, the oligonucleotide comprises a chemical modification.

In some embodiments, the oligonucleotide further comprises one or more nucleotides to each end of the sequence of 5′-(CCT)ₙ-3′.

In some embodiments, the oligonucleotide is administered through the route of orall, enteral, parenteral, or topical administration, or inhalation.

In some embodiments, the oligonucleotide is administered in combination with a Btk inhibitor, a PI3Kδ inhibitor, an IRAK inhibitor, an anti-CD20 monoclonal antibody, a SYK inhibitor, or a Bcl-2 inhibitor.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts that OCI-Ly3.3 was confirmed for the presence of MYD88 L265P mutant (FIG. 1, right panel) and OCI-Ly19 does not carried this mutant (FIG. 1, left panel). In consistence with previous report, OCI-Ly3.3 has homozygous MYD88 L265P mutant.

FIG. 2 depicts that 0.3 uM and 1 uM of the three TLR7/9 antagonists, referred as (CCT)₈, (CCT)₁₂ and (CCT)_{12M}, led to a cell growth inhibition on OCI-Ly3.3 but not OCI-Ly19 cells.

FIG. 3 depicts that all the three TLR7/9 antagonists were able to inhibit IL-10 secretion from OCI-Ly3.3 cells.

### DETAILED DESCRIPTION OF THE INVENTION

Several aspects of the invention are described below with reference to example applications for illustration. It should be understood that numerous specific details, relationships, and methods are set forth to provide a full understanding of the invention. One having ordinary skill in the relevant art, however, will readily recognize that the invention can be practiced without one or more of the specific details or with other methods. The present invention is not limited by the illustrated ordering of acts or events, as some acts may occur in different orders and/or concurrently with other acts or events.

Furthermore, not all illustrated acts or events are required to implement a methodology in accordance with the present invention.

The terminology used herein is for the purpose of describing particular embodiments only and is not intended to be limiting of the invention. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

### I. Definitions and Abbreviations

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry and nucleic acid chemistry and hybridization are those well known and commonly employed in the art. Standard techniques are used for nucleic acid and peptide synthesis. The techniques and procedures are generally performed according to conventional methods in the art and various general references, which are provided throughout this document. The nomenclature used herein and the laboratory procedures in analytical chemistry, and organic synthetic described below are those well-known and commonly employed in the art. Standard techniques, or modifications thereof, are used for chemical syntheses and chemical analyses.

### II. The Compositions

The present invention provides methods and compositions for treating cancers, such as B-cell lymphoma in a subject that has been diagnosed as having a B-cell lymphoma, preferably characterized by a mutation in MYD88 and is in need of such treatment by administering to the subject a pharmaceutical composition comprising a therapeutically effective amount of an oligonucleotide that is capable of inhibiting TRL9.

The oligonucleotides of the present invention strongly inhibits TLR9 activation. CpG containing oligonucleotides (CpG ODN) is known as a TLR9 agonist [D. M. Klinman, Nat. Rev., Immunol. 4 (2004) 249-258].

By “Oligonucleotide” herein is meant means multiple nucleotides (i.e. molecules comprising a sugar (e.g. deoxyribose) linked to a phosphate group and to an exchangeable organic base, which is either a substituted pyrimidine (Py) (e.g., cytosine (C), thymine (T)) or a substituted purine (Pu) (e.g., adenine (A) or guanine (G)). The term oligonucleotide as used herein refers to oligodeoxyribonucleotide (ODN). The oligonucleotide can be obtained from existing nucleic acid sources (e.g., genomic or cDNA), but are preferably synthetic. The oligonucleotide of the invention can be synthesized by a variety of automated nucleic acid synthesizers available in the market. These oligonucleotides are referred to as synthetic oligonucleotides.

It has been documented that TLR9 agonist activates both innate and adaptive immune response (Arthur M. Krieg. Nature Reviews Drug Discovery, Vol 5. June 2006, 471-484). CpG containing oligonucleotides (CpG ODN) is a TLR9 agonist [D. M. Klinman, Nat. Rev., Immunol. 4 (2004) 249-258]. Based on the functional characteristics, CpG ODNs are divided into three types (Tomoki Ito, et al. Blood, 2006, Vol 107, Num 6: 2423-2431). A-type CpG ODN activates human plasmacytoid dendritic cells (pDCs) to produce large amount of type I interferon (IFN-a/β) and strongly activates natural killer cells (NK cells). B-type CpG ODN primarily activates B cells, resulting in their proliferation and antibody secretion. C-type CpG ODN shares the activities of both A- and B-type CpG ODN. As a TLR9 agonist, CpG ODN such as CpG 2216 or CpG 2006 or CpG 2395 can be endocytosed into a cellular compartment where they are exposed to and activate TLR9. In pDC, TLR9 activation initiate a rapid innate immune response that is characterized by the secretion of pro-inflammatory cytokines [IL-6, tumor-necrosis factor-α (TNFα)], the secretion of type I interferon (IFN) and the secretion of secretion of IFN-inducible chemokines. Through both IFN-dependent and IFN-independent pathways, innate immune cells including natural killer (NK) cells, monocytes and neutrophils are secondarily activated by the pDC. B cells activated through TLR9 have a greatly increased sensitivity to antigen stimulation and efficiently differentiate into antibody-secreting cells, and therefore contributing to the adaptive immune response, especially humoral immune response. pDC activated through TLR9 secrete IFNα, which drives the migration and clustering of pDC to lymph nodes and other secondary lymphoid tissues where the pDC activates naive and memory T cells, assists the cross-presentation of soluble protein antigens to CD8+ cytotoxic T lymphocyte (CTL) and promotes strong TH1 biased cellular CD4 and CD8 T-cell responses. Based on the above mentioned findings, it is obvious that the agents that antagonize the activity of CpG ODN can be used to treat or prevent the immune-mediated disorder by inhibiting both innate and adaptive immune response.

In general, the method comprises administering to the subject a pharmaceutical composition comprising a therapeutically effective amount of an oligonucleotide, such as one having a sequence of 5′-(CCT)ₙ-3′, wherein n is an integer from 2 to 50, inclusive, and optionally, a pharmaceutically acceptable carrier.

In some embodiments, the oligonucleotide the oligonucleotide further comprises one or more nucleotides to each end of the sequence of 5′-(CCT)ₙ-3′.

In some embodiments, the oligonucleotide having a sequence of 5′-(CCT)nCm-3, n is an integer from 6 to 16, and m is 0, 1, or 2.

In some embodiments, the oligonucleotide comprises a sequence selected from the group consisting of:

5′-cctcctcctcctcctcctcctcctc-3′ (SEQ ID NO: 1),
5′-cctcctcctcctcctcctcctcctcc-3′ (SEQ ID NO.: 2),
5′-cctcctcctcctcctcctcctcctcct-3′ (SEQ ID NO.: 3),
5′-cctcctcctcctcctcctcctcctcctc-3′ (SEQ ID NO.: 4),
5′-cctcctcctcctcctcctcctcctcctcc-3′ (SEQ ID NO.: 5),
5′-cctcctcctcctcctcctcctcctcctcct-3′ (SEQ ID NO.: 6),
5′-cctcctcctcctcctcctcctcctcctcctc-3′ (SEQ ID NO.: 7),
5′-cctcctcctcctcctcctcctcctcctcctcc-3′ (SEQ ID NO.: 8),
5′-cctcctcctcctcctcctcctcctcctcctcct-3′ (SEQ ID NO.: 9),
5′-cctcctcctcctcctcctcctcctcctcctcctc-3′ (SEQ ID NO.: 10),
5′-cctcctcctcctcctcctcctcctcctcctcctcc-3′ (SEQ ID NO.: 11),
5′-cctcctcctcctcctcctcctcctcctcctcctcct-3′ (SEQ ID NO.: 12),
5′-cctcctcctcctcctcctcctcctcctcctcctcctcctcct-3′ (SEQ ID NO.: 13),
5′-cctcctcctcctcctcctcctcctcctcctcctcctcctcctcctcct-3′ (SEQ ID NO.: 14),
5′-cctcctcctcctcctcct-3′ (SEQ ID NO.: 15), and
5′-cctcctcctcctcctcctcct-3′ (SEQ ID NO.: 16). In these ODNs, bases are either unmodified or are
chemically modified, such as phosphorothioate-modified.

In some embodiments, the oligonucleotides of the present invention does not comprise a chemical modification.

In some embodiments, the oligonucleotides of the present invention comprise a chemical modification.

The oligonucleotide disclosed in the invention can encompass various chemical modifications, in comparison to natural DNA, involving a phosphodiester internucleoside bridge, a ribose unit and/or a natural nucleoside base (adenine, guanine, cytosine, and thymine). The modifications can occur either during or after synthesis of the oligonucleotide. During the synthesis, modified bases can be incorporated internally or on its end. After the synthesis, the modification can be carried out using the active groups (via an amino modifier, via the 3′ or 5′ hydroxyl groups, or via the phosphate group).

An oligonucleotide according to the invention may have one or more modifications, wherein each modification is located at a particular phosphodiester internucleoside bridge and/or at a particular ribose unit and/or at a particular natural nucleoside base position in comparison to an oligonucleotide of the same sequence, which is composed of natural DNA. The chemical modification includes “back bone modification” of the oligonucleotide of the invention. As used herein, the modified back bone of the oligonucleotide of the invention includes, but not limited to the “phosphorothioate backbone” that refers to a stabilized sugar phosphate backbone of a nucleic acid molecule in which a non-bridging phosphate oxygen is replaced by sulfur at least one internucleotide linkage.

In some embodiments a non-bridging phosphate oxygen is replaced by sulfur at each and every internucleotide linkage. Other back bone modifications denote the modification with nonionic DNA analogs, such as alkyl- and aryl-phophonates (in which the charged phosphonate oxygen is replaced by an alkyl or aryl group), phophodiester and alkylphosphotriesters, in which the charged oxygen moiety is alkylated.

In some embodiments, the phosphate backbone of the oligonucleotide is unmodified.

In some embodiments phosphate backbone of the oligonucleotide is partially or completely phosphorothioate-modified.

In some embodiments, the oligonucleotide comprises a sequence selected from the group consisting of:

### 5′-CCtCCTCCTCCTCCTCCTCCTCCTCCTCCTCCTCCT-3′ (SEQ ID NO.: 21),

### 5′-CCtCCtCCTCCTCCTCCTCCTCCTCCTCCTCCTCCT-3′ (SEQ ID NO.: 22),

### 5′-CCtCCtCCtCCTCCTCCTCCTCCTCCTCCTCCTCCT-3′ (SEQ ID NO.: 23),

### 5′-CCtCCtCCtCCtCCTCCTCCTCCTCCTCCTCCTCCT-3′ (SEQ ID NO.: 24),

### 5′-CCtCCtCCtCCtCCCCTCtCTCCTCCTCCTCCTCCT-3′ (SEQ ID NO.: 25),

### 5′-CCtCCtCCtCCtCCtCCtCCTCCTCCTCCTCCTCCT-3′ (SEQ ID NO.: 26),

### 5′-CCtCCtCCtCCtCCtCCtCCtCCTCCTCCTCCTCCT-3′ (SEQ ID NO.: 27),

### 5′-CCtCCtCCtCCtCCtCCtCCtCCtCCTCCTCCTCCT-3′ (SEQ ID NO.: 28),

### 5′-CCtCCtCCtCCtCCtCCtCCtCCtCCTCCTCCTCCT-3′ (SEQ ID NO.: 29),

### 5′-CCtCCtCCtCCtCCtCCtCCtCCtCCtCCTCCTCCT-3′ (SEQ ID NO.: 30),

### 5′-CCtCCtCCtCCtCCtCCtCCtCCtCCtCCtTCCTCCT-3′ (SEQ ID NO.: 31),

### 5′-CCtCCtCCtCCtCCtCCtCCtCCtCCtCCtCCtCCT-3′ (SEQ ID NO.: 32),

### 5′-CCtCCtCCtCCtCCtCCtCCtCCtCCtCCtCCtCCt-3′ (SEQ ID NO.: 33),

### 5′-CCtCCTCCTCCTCCTCCtCCTCCTCCTCCTCCTCCT-3′ (SEQ ID NO.: 34),

### 5′-CCTCCtCCTCCTCCTCCTCCTCCtCCTCCTCCTCCT-3′ (SEQ ID NO.: 35),

### 5′-CCTCCTCCtCCTCCTCCTCCTCCTCCtCCTCCTCCT-3′ (SEQ ID NO.: 36),

### 5′-CCTCCTCCTCCTCCtCCTCCTCCTCCTCCTCCtCCT-3′ (SEQ ID NO.: 37),

### 5′-CCtCCTCCTCCTCCtCCTCCTCCTCCtCCTCCTCCT-3′ (SEQ ID NO.: 38),

### 5′-CCTCCtCCTCCTCCTCCtCCTCCTCCTCCtCCTCCT-3′ (SEQ ID NO.: 39),

### 5′-CCTCCTCCtCCTCCTCCTCCtCCTCCTCCTCCtCCT-3′ (SEQ ID NO.: 40),

### 5′-CCTCCTCCTCCtCCTCCTCCTCCtCCTCCTCCTCCt-3′ (SEQ ID NO.: 41),

### 5′-CCtCCTCCTCCtCCTCCTCCtCCTCCTCCtCCTCCT-3′ (SEQ ID NO.: 42),

### 5′-CCTCCtCCTCCTCCtCCTCCTCCtCCTCCTCCtCCT-3′ (SEQ ID NO.: 43),

### 5′-CCTCCTCCtCCTCCTCCtCCTCCTCCtCCTCCTCCt-3′ (SEQ ID NO.: 44),

### 5′-CCtCCTCCtCCTCCtCCTCCtCCTCCtCCTCCTCCT-3′ (SEQ ID NO.: 45),

### 5′-CCTCCTCCtCCTCCtCCTCCtCCTCCtCCtCCTCCT-3′ (SEQ ID NO.: 46),

### 5′-CCTCCTCCTCCTCCtCCTCCtCCTCCtCCtCCtCCT-3′ (SEQ ID NO.: 47),

### 5′-CCTCCTCCTCCTCCTCCTCCtCCTCCtCCtCCtCCt-3′ (SEQ ID NO.: 48),

### 5′-CCtCCTCCtCCTCCtCCTCCtCCTCCtCCTCCtCCT-3′(SEQ ID NO.: 49),

### 5′-CCTCCtCCTCCtCCTCCtCCTCCtCCTCCtCCTCCt-3′(SEQ ID NO.: 50),

wherein for SEQ ID NOs. 21-50, a capitol case letter denotes the base is phosphorothioate-modified, and lower case letter denotes that the bases the base is un-modified.

In some embodiments, the oligonucleotide is a phosphorothioate/phosphodiester chimera.

The chemical modification also includes the base substitutions of the oligonucleotide disclosed in the invention. The substituted purines and pyrimidines can be C-5 propyne pyrimidine and 7-deaza-7-substituted purine. The substituted purines and pyrimidines include but are not limited to adenine, cytosine, guanine, and thymine, and other naturally and non-naturally occurring nucleobases. The chemical modification of the oligonucleotide of the invention further includes the modification of the bases of the oligonucleotide. A modified base is any base which is chemically distinct from the naturally occurring bases typically found in DNA such as T, C, G and A, but which share basic chemical structures with these naturally occurring bases.

In some embodiments, the oligonucleotide of the invention is modified by using cytidine derivatives. The term “cytidine derivative” refers to a cytidine-like nucleotide (excluding cytidine) and the term “thymidine derivative” refers to a thymidine-like nucleotide (excluding thymidine). In addition, the oligonucleotides of the invention can be chemically modified by linking a diol, such as tetraethyleneglycol or hexaethyleneglycol, at either or both termini of the oligonucleotide.

The oligonucleotides may have further backbone modifications in addition to the phosphonoacetate or phosphonoacetate-like linkage at the Py-Pu dinucleotide. A stabilized internucleotide linkage is an internucleotide linkage that is relatively resistant to in vivo degradation (e.g., via an exo- or endo-nuclease), compared to a phosphodiester internucleotide linkage. In addition to the phosphonoacetate, and phosphonoacetate-like linkages, the oligonucleotides may contain other stabilized internucleotide linkages, including, without limitation, phosphorothioate, phosphorodithioate, methylphosphonate, and methylphosphorothioate. Other stabilized internucleotide linkages include, without limitation: peptide, alkyl, and dephospho. Phosphonoacetate internucleotide linkages, like other stabilized linkages, have reduced susceptibility to nuclease digestion and increased ability to activate RNAse H. Thus for example phosphodiester, but not phosphonoacetate, oligonucleotides are susceptible to nuclease digestion, while both phosphodiester and phosphonoacetate oligonucleotides activate RNAse H. In some embodiments, the Py-Pu oligonucleotide includes at least one phosphodiester internucleotide linkage. The oligonucleotides may include, in addition to the phosphonoacetate or phosphonoacetate-like internucleotide linkages at preferred internal positions, 5′ and 3′ ends that are resistant to degradation. Such degradation-resistant ends can involve any suitable modification that results in an increased resistance against exonuclease digestion over corresponding unmodified ends. For instance, the 5′ and 3′ ends can be stabilized by the inclusion there of at least one phosphate modification of the backbone. In one embodiment, the at least one phosphate modification of the backbone at each end is independently a phosphorothioate, phosphorodithioate, phosphonoacetate, phosphonoacetate-like, methylphosphonate, or methylphosphorothioate internucleotide linkage. In another embodiment, the degradation-resistant end includes one or more nucleotide units connected by peptide or amide linkages at the 3′ end.

The terms “nucleic acid” and “oligonucleotide” also encompass nucleic acids or oligonucleotides with substitutions or modifications, such as in the bases and/or sugars. For example, they include nucleic acids having backbone sugars that are covalently attached to low molecular weight organic groups other than a hydroxyl group at the 2′ position and other than a phosphate group or hydroxy group at the 5′ position. Thus modified nucleic acids may include a 2′-O-alkylated deoxyribose group. In addition, modified nucleic acids may include sugars such as arabinose or 2′-fluoroarabinose instead of deoxyribose. Thus the nucleic acids may be heterogeneous in backbone composition thereby containing any possible combination of polymer units linked together such as peptide-nucleic acids (which have an amino acid backbone with nucleic acid bases). In the context of the instant invention, the oligonucleotides are not antisense oligonucleotides, ribozymes, or aptamers.

Nucleic acids also include substituted purines and pyrimidines such as C-5 propyne pyrimidine and 7-deaza-7-substituted purine modified bases (Wagner R W et al., (1996) Nat Biotechnol 14:840-4). Purines and pyrimidines include but are not limited to adenine, cytosine, guanine, thymine, 5-methylcytosine, 5-hydroxycytisine, 5-fluorocytosine, 2-aminopurine, 2-amino-6-chloropurine, 2,6-diaminopurine, hypoxanthine, and other naturally and non-naturally occurring nucleobases, substituted and unsubstituted aromatic moieties. Other such modifications are well known to those of skill in the art.

The oligonucleotides may be DNA or RNA. In one embodiment the oligonucleotides of the invention are DNA/RNA hybrid molecules comprising a mixed backbone of ribose and deoxyribose. DNA/RNA hybrid oligonucleotides often demonstrate increased activities. In one embodiment these DNA/RNA hybrid oligonucleotides are single-stranded. In another embodiment all or part of the oligonucleotide is double-stranded. In one embodiment the oligonucleotides of the invention are in the form of covalently closed, dumbbell-shaped molecules with both primary and secondary structure. In one embodiment such cyclic oligoribonucleotides include two single-stranded loops connected by an intervening double-stranded segment. In one embodiment at least one single-stranded loop includes an immunostimulatory DNA motif of the invention. Other covalently closed, dumbbell-shaped molecules of the invention include chimeric DNA/RNA molecules in which, for example, the double-stranded segment is at least partially DNA (e.g., either homodimeric dsDNA or heterodimeric DNA:RNA) and at least one single-stranded loop includes an immunostimulatory DNA motif of the invention. Alternatively, the double stranded segment of the chimeric molecule is DNA.

The oligonucleotides of the invention can also include other modifications. These include nonionic DNA analogs, such as alkyl- and aryl-phosphates (in which the charged phosphonate oxygen is replaced by an alkyl or aryl group), phosphodiester and alkylphosphotriesters, in which the charged oxygen moiety is alkylated. Nucleic acids which contain diol, such as tetraethyleneglycol or hexaethyleneglycol, at either or both termini have also been shown to be substantially resistant to nuclease degradation.

The oligonucleotides of the instant invention can encompass various chemical modifications and substitutions, in comparison to natural RNA and DNA, involving a phosphodiester internucleotide bridge, a β-D-ribose unit and/or a natural nucleotide base (adenine, guanine, cytosine, thymine, uracil). Examples of chemical modifications are known to the skilled person and are described, for example, in Uhlmann, E. et al., (1990) Chem Rev 90:543; “Protocols for Oligonucleotides and Analogs” Synthesis and Properties & Synthesis and Analytical Techniques, S. Agrawal, Ed, Humana Press, Totowa, USA 1993; Crooke, S T. et al., (1996) Annu Rev Pharmacol Toxicol 36:107-129; and Hunziker, J. et al., (1995) Mod Synth Methods 7:331-417. An oligonucleotide according to the invention may have one or more modifications, wherein each modification is located at a particular phosphodiester internucleotide bridge and/or at a particular β-D-ribose unit and/or at a particular natural nucleotide base position in comparison to an oligonucleotide of the same sequence which is composed of natural DNA or RNA.

For example, the invention relates to an oligonucleotide which may comprise one or more modifications and wherein each modification is independently selected from: a) the replacement of a phosphodiester internucleotide bridge located at the 31 and/or the 51 end of a nucleotide by a modified internucleotide bridge, b) the replacement of phosphodiester bridge located at the 3′ and/or the 5′ end of a nucleotide by a dephospho bridge, c) the replacement of a sugar phosphate unit from the sugar phosphate backbone by another unit, d) the replacement of a β-D-ribose unit by a modified sugar unit, and e) the replacement of a natural nucleotide base by a modified nucleotide base.

A phosphodiester internucleotide bridge located at the 3′ and/or the 5′ end of a nucleotide can be replaced by a modified internucleotide bridge, wherein the modified internucleotide bridge is for example selected from phosphorothioate, phosphorodithioate, NR1R2-phosphoramidate, boranophosphate, α-hydroxybenzyl phosphonate, phosphate-(CrC2i)-O-alkyl ester, phosphate-[(C6-Ci2)aryl-(Ci-C2i)-O-alkyljester, (d-CeJalkylphosphonate and/or
(C₆-C₁₂)arylphosphonate
   bridges, (C7-C12)-α-hydroxymethyl-aryl (e.g., disclosed in WO 95/01363), wherein (C6-Ci2)aryl, (Cβ-C2o)aryl and (C6-Ci4)aryl are optionally substituted by halogen, alkyl, alkoxy, nitro, cyano, and where R1 and R2 are, independently of each other, hydrogen, (CrCi8)-alkyl, (C6-C2o)-aryl, (C6-Ci4)-aryl-(CrC8)-alkyl, preferably hydrogen, (CrC8)-alkyl, preferably (Ci-C4)-alkyl and/or methoxyethyl, or R1 and R2 form, together with the nitrogen atom carrying them, a 5-6-membered heterocyclic ring which can additionally contain a further heteroatom from the group O, S and N.

The replacement of a phosphodiester bridge located at the 3′ and/or the 5′ end of a nucleotide by a dephospho bridge (dephospho bridges are described, for example, in Uhlmann E and Peyman A in “Methods in Molecular Biology,” Vol. 20, “Protocols for Oligonucleotides and Analogs,” S. Agrawal, Ed., Humana Press, Totowa 1993, Chapter 16, pp. 355 ff), wherein a dephospho bridge is for example selected from the dephospho bridges formacetal, 3′-thioformacetal, methylhydroxylamine, oxime, methylenedimethyl-hydrazo, dimethylenesulfone and/or silyl groups. A sugar phosphate unit (i.e., a β-D-ribose and phosphodiester internucleotide bridge together forming a sugar phosphate unit) from the sugar phosphate backbone (i.e., a sugar phosphate backbone is composed of sugar phosphate units) can be replaced by another unit, wherein the other unit is for example suitable to build up a “morpholino-derivative” oligomer (as described, for example, in Stirchak E P et al., (1989) Nucleic Acids Res 17:6129-41), that is, e.g., the replacement by a morpholino-derivative unit; or to build up a polyamide nucleic acid (“PNA”; as described for example, in Nielsen P E et al., (1994) Bioconjug Chem 5:3-7), that is, e.g., the replacement by a PNA backbone unit, e.g., by 2-aminoethylglycine.

A β-D-ribose unit or a β-D-2′-deoxyribose unit can be replaced by a modified sugar unit, wherein the modified sugar unit is for example selected from α-D-2′-deoxyribose, α-L-2′-deoxyribose, β-L-2′-deoxyribose, β-L-ribose, 2′-F-21-deoxyribose, 2′-F-2′-deoxy-arabinose, 2′-O-(Ci-C6)alkyl-ribose, preferably 2′-O—(CrC6)alkyl-ribose is 2′-O-methylribose, 2I-O-(C2-C6)alkenyl-ribose, 21-[O-(Ci-C6)alkyl-O-(Ci-C6)alkyl]-ribose, 21-NH2-21-deoxyribose, β-D-xylo-furanose, α-arabinofuranose, 2,4-dideoxy-β-D-erythro-hexo-pyranose, and carbocyclic (described, for example, in Froehler, J. (1992) Am Chem Soc 114:8320) and/or open-chain sugar analogs (described, for example, in Vandendriessche et al., (1993) Tetrahedron 49:7223) and/or bicyclosugar analogs (described, for example, in Tarkov, M. et al., (1993) HeIv Chim Acta 76:481).

In some embodiments the sugar is 2′-O-methylribose, 2′-deoxyribose, 2′-fluoro-2′-deoxyribose, 2′-amino-2′deoxyribose, 2′-O-alkyl-ribose, or 3′-O-alkyl-ribose and/or 2′-O-4′-C-alkylene ribose, such as 2′-O-4′-C-methylene ribose (also called LNA).

Nucleic acids also include substituted purines and pyrimidines such as C-5 propyne pyrimidine and 7-deaza-7-substituted purine modified bases (Wagner, R. W. et al., (1996) Nat Biotechnol 14:840-4). Purines and pyrimidines include but are not limited to adenine, cytosine, guanine, and thymine, and other naturally and non-naturally occurring nucleobases, substituted and unsubstituted aromatic moieties.

A modified base is any base which is chemically distinct from the naturally occurring bases typically found in DNA and RNA such as T, C, G, A, and U, but which share basic chemical structures with these naturally occurring bases. The modified nucleotide base may be, for example, selected from hypoxanthine, uracil, dihydrouracil, pseudouracil, 2-thiouracil, 4-thiouracil, 5-aminouracil, 5-(Ci-C6)-alkyluracil, 5-(C2-Cβ)-alkenyluracil, 5-(C2-C6)-alkynyluracil, 5-(hydroxymethyl)uracil, 5-chlorouracil, 5-fluorouracil, 5-bromouracil, 5-iodo-uracil, 2,4-difluoro-toluene, and 3-nitropyrrole, 5-hydroxycytosine, 5-(CrC6)-alkylcytosine, 5-(C2-C6)-alkenylcytosine, 5-(C2-Ce)-alkynylcytosine, 5-chlorocytosine, 5-fluorocytosine, 5-bromocytosine, N2-dimethylguanine, 2,4-diamino-purine, 8-azapurine, a substituted 7-deazapurine, preferably 7-deaza-7-substituted and/or 7-deaza-8-substituted purine, 5-hydroxymethylcytosine, N4-alkylcytosine, e.g., N4-ethylcytosine, 5-hydroxydeoxycytidine, 5-hydroxymethyldeoxycytidine, N4-alkyldeoxycytidine, e.g., N4-ethyldeoxycytidine, 6-thiodeoxyguanosine, and deoxyribonucleotides of nitropyrrole, C5-propynylpyrimidine, and diaminopurine e.g., 2,6-diaminopurine, inosine, 5-methylcytosine, 2-aminopurine, 2-amino-6-chloropurine, hypoxanthine or other modifications of a natural nucleotide bases. This list is meant to be exemplary and is not to be interpreted to be limiting.

Herein “Py” is used to refer to pyrimidine and in some embodiments a nucleotide containing a cytosine or a modified cytosine. A modified cytosine as used herein is a naturally occurring or non-naturally occurring pyrimidine base analog of cytosine which can replace this base without impairing the immunostimulatory activity of the oligonucleotide. Modified cytosines include but are not limited to 5-substituted cytosines (e.g., 5-methyl-cytosine, 5-fluoro-cytosine, 5-chloro-cytosine, 5-bromocytosine, 5-iodo-cytosine, 5-hydroxy-cytosine, 5-hydroxymethyl-cytosine, 5-difluoromethyl-cytosine, and unsubstituted or substituted 5-alkynyl-cytosine), 6-substituted cytosines, N4-substituted cytosines (e.g., N4-ethyl-cytosine), 5-aza-cytosine, 2-mercapto-cytosine, isocytosine, pseudo-isocytosine, cytosine analogs with condensed ring systems (e.g., N,N′-propylene cytosine or phenoxazine), and uracil and its derivatives (e.g., 5-fluoro-uracil, 5-bromo-uracil, 5-bromovinyl-uracil, 4-thio-uracil, 5-hydroxy-uracil, 5-propynyl-uracil). Some of the preferred cytosines include 5-methylcytosine, 5-fluoro-cytosine, 5-hydroxy-cytosine, 5-hydroxymethyl-cytosine, and N4-ethyl-cytosine. In another embodiment of the invention, the cytosine base is substituted by a universal base (e.g., 3-nitropyrrole, P-base), an aromatic ring system (e.g., fluorobenzene or difluorobenzene) or a hydrogen atom (dSpacer).

Herein “Pu” is used to refer to a purine or modified purine. In some embodiments Pu is a guanine or a modified guanine base. A modified guanine as used herein is a naturally occurring or non-naturally occurring purine base analog of guanine which can replace this base without impairing the immunostimulatory activity of the oligonucleotide. Modified guanines include but are not limited to 7-deazaguanine, 7-deaza-7-substituted guanine (such as 7-deaza-7-(C2-C6)alkynylguanine), 7-deaza-8-substituted guanine, hypoxanthine, N2-substituted guanines (e.g., N2-methyl-guanine), 5-amino-3-methyl-3H,6H-thiazolo [4,5-d]pyrimidine-2,7-dione, 2,6-diaminopurine, 2-aminopurine, purine, indole, adenine, substituted adenines (e.g., N6-methyl-adenine, 8-hydroxyadenine) 8-substituted guanine (e.g., 8-hydroxyguanine and 8-bromoguanine), and 6-thioguanine. In another embodiment of the invention, the guanine base is substituted by a universal base (e.g., 4-methyl-indole, 5-nitro-indole, and K-base), an aromatic ring system (e.g., benzimidazole or dichloro-benzimidazole, 1-methyl-1H-[1,2,4]triazole-3-carboxylic acid amide) or a hydrogen atom (dSpacer).

The invention also encompasses oligonucleotides having unusual internucleotide linkages, including 5′-5′, 2′-2′, 2′-3′, and 2′-5′ internucleotide linkages. In some aspects of the invention it is advantageous for the oligonucleotides to have one or more accessible 51 ends. It is possible to create modified oligonucleotides having two such 51 ends. This may be achieved, for instance by attaching two oligonucleotides through a 3′-3′ linkage to generate an oligonucleotide having one or two accessible 51 ends. The 3′-3′ linkage may be a phosphodiester, phosphorothioate, phosphonoacetate or any other modified internucleotide bridge. Methods for accomplishing such linkages are known in the art. For instance, such linkages have been described in Seliger, H. et al. Oligonucleotide analogs with terminal 3′-3′- and 5′-5′-internucleotidic linkages as antisense inhibitors of viral gene expression, Nucleotides & Nucleotides (1991), 10(1-3), 469-77 and Jiang et al., Pseudo-cyclic oligonucleotides: in vitro and in vivo properties, Bioorganic & Medicinal Chemistry (1999), 7(12), 2727-2735. In one embodiment such unusual linkages are excluded from the immunostimulatory DNA motif, even though one or more of such linkages may occur elsewhere within the polymer. For polymers having free ends, inclusion of one 3′-3′ internucleotide linkage can result in a polymer having two free 5′ ends. Conversely, for polymers having free ends, inclusion of one 5-5′ internucleotide linkage can result in a polymer having two free 3′ ends. Additionally, 3′3′-, 5-5′-, 2′-2′-, 2′-3′-, and 2′-5′-linked nucleic acids where the linkage is not a phosphodiester, phosphorothioate, phosphonoacetate or other modified bridge, can be prepared using an additional spacer, such as tri- or tetra-ethylenglycol phosphate moiety (Durand, M. et al., Triple-helix formation by an oligonucleotide containing one (dA)12 and two (dT)12 sequences bridged by two hexaethylene glycol chains, Biochemistry (1992), 31(38), 9197-204, U.S. Pat. No. 5,658,738, and U.S. Pat. No. 5,668,265). Alternatively, the non-nucleotidic linker may be derived from ethanediol, propanediol, or from an abasic deoxyribose (dSpacer) unit (Fontanel, Marie Laurence et al., Sterical recognition by T4 polynucleotide kinase of non-nucleosidic moieties 51-attached to oligonucleotides; Nucleic Acids Research (1994), 22(11), 2022-7) using standard phosphoramidite chemistry. The non-nucleotidic linkers can be incorporated once or multiple times, or combined with each other allowing for any desirable distance between the 3′-ends of the two ODNs to be linked.

The oligonucleotide may contain a doubler or trebler unit (Glen Research, Sterling, Va.), in particular those modified oligodeoxyribonucleotide analogs with a 3′-3′ linkage. A doubler unit in one embodiment can be based on 1,3-bis-[5-(4,4′-dimethoxytrityloxy)pentylamido]propyl-2-[(2-cyanoethyl)-(N,N-diisopropyl)]-phosphoramidite. A trebler unit in one embodiment can be based on incorporation of Tris-2,2,2-[3-(4,4′-dimethoxytrityloxy)propyloxymethyl]ethyl-[(2-cyanoethyl)-(N,N-diisopropyl)]-phosphoramidite. Branching of the modified oligoribonucleotide analogs by multiple doubler, trebler, or other multiplier units leads to dendrimers which are a further embodiment of this invention. Branched modified oligoribonucleotide analogs may lead to crosslinking of receptors particularly for combinations of immunostimulatory RNA and DNA such as TLR3, TLR7, TLR8, and TLR9 with distinct immune effects compared to non-branched forms of the analogs. In addition, the synthesis of branched or otherwise multimeric analogs may stabilize DNA against degradation and may enable weak or partially effective DNA sequences to exert a therapeutically useful level of immune activity. The modified oligodeoxyribonucleotide analogs may also contain linker units resulting from peptide modifying reagents or oligonucleotide modifying reagents (Glen Research). Furthermore, the modified oligodeoxyribonucleotide analogs may contain one or more natural or unnatural amino acid residues which are connected to the polymer by peptide (amide) linkages.

In some embodiments, the oligonucleotides can be modified with the linking of one or more polyethylene glycol (PEG) chains. The PEGylated oligonucleotide can prolong the circulation time in vivo by reducing renal clearance.

The 3′-5′, 5′-5′, 3′-3\ 2′-2′, 2′-3′, and 2′-5′ internucleotide linkages can be direct or indirect. Direct linkages in this context refers to a phosphate or modified phosphate linkage as disclosed herein, without an intervening linker moiety. An intervening linker moiety is an organic moiety distinct from a phosphate or modified phosphate linkage as disclosed herein, which can include, for example, polyethylene glycol, triethylene glycol, hexaethylene glycol, dSpacer (i.e., an abasic deoxynucleotide), doubler unit, or trebler unit. The linkages are preferably composed of C, H, N, O, S, B, P, and Halogen, containing 3 to 300 atoms. An example with 3 atoms is an acetal linkage (ODN1-3′-O— CH2-O-3′-ODN2) connecting e.g., the 3′-hydroxy group of one nucleotide to the 3′-hydroxy group of a second oligonucleotide. An example with about 300 atoms is PEG-40 (tetraconta polyethyleneglycol). Preferred linkages are phosphodiester, phosphorothioate, methylphosphonate, phosphoramidate, boranophosphonate, amide, ether, thioether, acetal, thioacetal, urea, thiourea, sulfonamide, Schiff Base and disulfide linkages. It is also possible to use the Solulink BioConjugation System.

If the oligonucleotide is composed of two or more sequence parts, these parts can be identical or different. Thus, in an oligonucleotide with a 3′3′-linkage, the sequences can be identical 5′-ODN1-3′3′-ODN1-5′ or different 5′-ODN1-3′3′-ODN2-5′. Furthermore, the chemical modification of the various oligonucleotide parts as well as the linker connecting them may be different. Since the uptake of short oligonucleotides appears to be less efficient than that of long oligonucleotides, linking of two or more short sequences results in improved immune stimulation. The length of the short oligonucleotides is preferably 2-20 nucleotides, more preferably 3-16 nucleotides, but most preferably 5-10 nucleotides. Preferred are linked oligonucleotides which have two or more unlinked 5′-ends.

The oligonucleotide partial sequences may also be linked by non-nucleotidic linkers. A “non-nucleotidic linker” as used herein refers to any linker element that is not a nucleotide or polymer thereof (i.e., a polynucleotide), wherein a nucleotide includes a purine or pyrimidine nucleobase and a sugar phosphate, in particular abasic linkers (dSpacers), trietyhlene glycol units or hexaethylene glycol units. Further preferred linkers are alkylamino linkers, such as C3, C6, C12 aminolinkers, and also alkylthiol linkers, such as C3 or C6 thiol linkers. The oligonucleotides can also be linked by aromatic residues which may be further substituted by alkyl or substituted alkyl groups.

Other stabilized oligonucleotides include: nonionic DNA analogs, such as alkyl- and aryl-phosphates (in which the charged phosphonate oxygen is replaced by an alkyl or aryl group), phosphodiester and alkylphosphotriesters, in which the charged oxygen moiety is alkylated. Nucleic acids which contain diol, such as tetraethyleneglycol or hexaethyleneglycol, at either or both termini have also been shown to be substantially resistant to nuclease degradation.

### III. B-Cell Lymphoma Characterized by L265P Mutation of MYD88

In one aspect, the present invention provides methods and compositions for treatment of cancer, such as B-cell lymphoma in a subject that has been diagnosed as having a B-cell lymphoma, preferally characterized by a mutation in MYD88 and is in need of such treatment.

As used herein, the term “subject” refers to an animal. Preferably, the animal is a mammal. A subject also refers to for example, primates (e.g., humans), cows, sheep, goats, horses, dogs, cats, rabbits, rats, mice, fish, birds and the like. In a preferred embodiment, the subject is a human.

Certain genetically defined B-cell lymphomas are those harboring oncogenic mutations in MYD88 in several types of B-cell lymphoma, including, but not limited to, Waldenstrom's macroglobulinemia (WM), the activated B-cell-like (ABC) subtype of diffuse large B-cell lymphoma (DLBCL), and gastric mucosa-associated lymphoid tissue (MALT) lymphoma.

By “agonist” herein is meant a substance that binds to a receptor of a cell and induces a response. An agonist often mimics the action of a naturally occurring substance such as a ligand.

By “antagonist” herein is meant substance that attenuates the effects of an agonist.

The oligonucleotides of the invention strongly inhibits the cytokines stimulated by CpG ODN, indicating that the oligonucleotides of the invention can be used as a remedy for the treatment of diseases related to TLR9 activation, such as B-cell lymphoma, specially certain genetically defined B-cell lymphomas harboring oncogenic mutations in MYD88, including Waldenstrom's macroglobulinemia (WM), the activated B-cell-like (ABC) subtype of diffuse large B-cell lymphoma (DLBCL), and gastric mucosa-associated lymphoid tissue (MALT) lymphoma.

TLRs have been implicated in the pathogenesis of B-cell lymphomas harboring oncogenic mutations in Myeloid differentiation primary response gene 88 (MYD88) (Lim K H et al. 2013 AACR Abs.). MYD88 is an adaptor molecule in TLR and interleukin 1 receptor (IL-1R) signaling. Following TLR or IL-1R stimulation, MYD88 is recruited to the activated receptor complex as a homodimer, which complexes with IL-1R-associated kinase (IRAK) 4 to activate IRAK1 (Fitzgerald K A et al. Nature. 2001 413 78-83). The activated IRAK1 binds with tumor necrosis factor receptor-associated factor 6 (TRAF6), which catalyzes the addition of polyubiquitin onto TRAF6. The addition of ubiquitin activates the TAK/TAB comples, which in turn phosphorylates interferon regulating factor (IRFs), resulting in nuclear factor κB (NF-κB) release and transport to the nucleus. NF-κB in the nucleus induces the expression of proinflammatory genes. Recently, oncogenic mutations in MYD88 have been identified in several types of B-cell lymphoma (Ngo V et al. Nature. 2011 115-119; Treon S P et al. NEJM. 2012 826-833; Poulain S et al. Blood. 2013 4504-4511). The existence of somatic L265P mutation in MYD88 has been observed with a higher frequency in WM (91%), ABC DLBCL (30%), and MALT lymphomas (9%). At a lower frequency, additional mutations were also observed in MYD88, occurring in both the ABC and germinal centre B-cell-like (GCB) DLBCL subtypes (Ngo V et al. Nature. 2011 470:115-119).

It has been reported that survival of MYD88 L265P-expressing ABC DLBCL cells was sustained by the mutant (Yang G et al. 2013 Blood 1222-1232). L265P mutant promoted the survival ABC DLBCL cells by spontaneously assembling a protein complex containing IRAK1 and IRAK4, leading to IRAK4 kinase activity, IRAK1 phosphorylation, NF-κB signalling, JAK kinase activation of STAT3, and secretion of IL-6, IL-10 and interferon-β (Ngo V et al. Nature. 2011 470:115-119). These studies demonstrate that L265P is a gain-of-function driver mutation and that an aberrant MYD88 signalling pathway is integral to the pathogenesis of WM and ABC DLBCL.

MYD88 L265P mutation has been shown to engage TLR7 and TLR9 to confer a survival benefit to ABC DLBCL cells (Ngo V et al. Nature. 2011 470:115-119). MyD88 L265P oncoprotein binds constitutively to TLR7 and TLR9, thereby amplifying signals that emanate from these receptors. Knockdown of TLR7 or TLR9 potently suppressed NF-κB activity in ABC DLBCL cell lines and promoted apoptosis. Depletion of known proteins essential for TLR7 and TLR9 trafficking and signaling, including UNC93B1, PRAT4A or CD14, was lethal for ABC DLBCL lines.

Pharmacologic inhibition of TLR7 and TLR9 function in lysosomal compartments using the cathepsin inhibitor zFA-fmk or hydroxychloroquine diminished MyD88 signaling and reduced survival of ABC DLBCL lines (Lin K H et al. Cancer Research 2013 Vol. 73(8) Abstract 2332. TLR7 or TLR9 mutants that are defective in ligand binding failed to promote the survival of ABC DLBCL lines following knockdown of endogenous TLR7 or TLR9 expression in ABC DLBCL lines (Poulain S et al Blood 2013 4504-4511).

Inhibition of TLR7 and TLR9 by inhibitory ODNs increased rates of cell death in tumor cells harboring MYD88 L265P mutation, inhibited cytokine production and key components of signaling pathways (Liang X Q et al. Blood 2010 5041-5052; Zhang Y S et al. Inter Immunophamarcology. 2012 446-453). In MYD88 L265P-expressing WM cell lines, inhibition of MYD88/IRAK signaling attenuates NF-κB signaling and tumor cell survival. Correspondingly, WM cell survival is enhanced by MYD88 L265P overexpression (Treon S P et al. NEJM. 2012 826-833; Ansell S M et al. Blood. 2012 2699; Poulain S et al. Blood. 2013 4504-11).

In some embodiments, the B-cell lymphoma is selected from the group consisting of: Waldenstrom's macroglobulinemia (WM), activated B-cell-like (ABC) subtype of diffuse large B-cell lymphoma (DLBCL), and gastric mucosa-associated lymphoid tissue (MALT) lymphoma.

In some embodiments, the mutation in MYD88 comprises L265P, M232T, S243N, or T294P.

It has been demonstrated that inhibition of specific TLRs is a useful approach to the treatment of certain genetically defined B-cell lymphomas (Lim K H et al. 2013 AACR Abs.). In these studies, a specific somatic L265P mutation has been identified in MYD88, an adaptor protein that mediates TLR and interleukin 1 receptor (IL-1R) signaling. The mutation has been shown to engage TLR7 and TLR9 to confer a survival benefit to the tumor cells, while inhibition of TLR7 and TLR9 led to increase rates of cell death in tumor cells harboring this mutation. The existence of L265P mutation in MYD88 has been observed with a higher frequency in 91% of Waldenström's macroglobulinemia (WM), 30% of the activated B-cell-like (ABC) subtype of diffuse large B-cell lymphoma (DLBCL), and 9% of gastric mucosa-associated lymphoid tissue (MALT) lymphomas. At a lower frequency, additional mutations were also observed in MYD88, occurring in both the ABC and germinal centre B-cell-like (GCB) DLBCL subtypes.

The oligonucleotides of the invention strongly inhibit cytokine production and key components of signaling pathways, increases rates of cell death in human lymphoma cell lines that carry the L265P mutation in MYD88. The oligonucleotides of the present invention can be used as a remedy for the treatment of certain genetically defined B-cell lymphomas that harbor oncogenic mutations in MYD88. They are included, but not limited to, Waldenstrom's macroglobulinemia (WM), the activated B-cell-like (ABC) subtype of diffuse large B-cell lymphoma (DLBCL), and gastric mucosa-associated lymphoid tissue (MALT) lymphoma.

The present invention provides methods and compositions for screening for cancer patients with MYD88 mutation using methods known in the art and/or provided herein, and treating such patients with an oligonucleotide as provided herein.

The MYD88 gene can be detected at genomic DNA level, mRNA level, or protein level. A biological sample from a subject in need of testing is obtained using methods known the art. The biological sample is optionally processed to obtain protein, RNA, and/or DNA, which is in turn used in assays to detect MYD88 mutation.

### A. Biological Sample

By “biological sample” herein is meant any biological sample suspected of containing MYD88 mutation polynucleotides or polypeptides or fragments thereof, and may comprise a cell, chromosomes isolated from a cell (e.g., a spread of metaphase chromosomes), genomic DNA (in solution or bound to a solid support such as for Southern analysis), RNA (in solution or bound to a solid support such as for northern analysis), cDNA (in solution or bound to a solid support), an extract from cells, blood, urine, marrow, or a tissue, and the like.

Biological samples useful in the practice of the methods of the invention may be obtained from any mammal in which a cancer characterized by the mutation of MYD88 is present or developing. In one embodiment, the mammal is a human. The human candidate may be a patient currently being treated with, or considered for treatment with, an oligonucleotide, such as those provided herein. In another embodiment, the mammal is large animal, such as a horse or cow, while in other embodiments, the mammal is a small animal, such as a dog or cat, all of which are known to develop cancers, including lung carcinomas.

Any biological sample comprising cells (or extracts of cells) from a mammalian cancer is suitable for use in the methods of the invention. Circulating tumor cells may also be obtained from serum using tumor markers, cytokeratin protein markers or other methods of negative selection as described (see Ma et al., Anticancer Res. 23(1A): 49-62 (2003)). Serum and bone marrow samples may be particularly preferred for patients with leukemia. For cancers involving solid tumors, such as sarcomas and carcinomas, the biological sample may comprise cells obtained from a tumor biopsy, which maybe be obtained according to standard clinical techniques.

Circulating tumor cells (“CTCs”) may be purified, for example, using the kits and reagents sold under the trademarks Vita-Assays™, Vita-Cap™, and CellSearch® (commercially available from Vitatex, LLC (a Johnson and Johnson corporation). Other methods for isolating CTCs are described (see, for example, PCT Publication No. WO/2002/020825, Cristofanilli et al., New Engl. J. of Med. 351 (8):781-791 (2004), and Adams et al., J. Amer. Chem. Soc. 130(27): 8633-8641 (July 2008)). In a particular embodiment, a circulating tumor cell (“CTC”) may be isolated and identified as having originated from the lung.

### B. Detection of MYD88 Mutation Polypeptide

In some embodiments, the MYD88 mutation (eg. MYD88 L265P) is detected by an immunoassay. An MYD88 L265P protein or peptide is generated to produce antibodies (monoclonal or polyclonal) specific for MYD88 L265P proteins. Such antibodies are then used in an assay to detect the presence of MYD88 L265P.

MYD88 L265P is generally detected using a Rspo fusion-specific reagent. By “MYD88 L265P-specific reagent” herein is meant any reagent, biological or chemical, capable of specifically binding to, detecting and/or quantifying the presence/level of expressed MYD88 L265P polypeptide in a biological sample. The term includes, but is not limited to, the preferred antibody and reagents discussed below, and equivalent reagents are within the scope of the present invention.

Reagents suitable for use in practice of the methods of the invention include an MYD88 L265P polypeptide-specific antibody. A MYD88 L265P-specific antibody of the invention is an isolated antibody or antibodies that specifically bind(s) a MYD88 L265P polypeptide of the invention (e.g. the peptide corresponding to the MYD88 L265P sequences provided herein) but does not substantially bind either wild type MYD88.

Human MYD88 L265P-specific antibodies may also bind to highly homologous and equivalent epitopic peptide sequences in other mammalian species, for example murine or rabbit, and vice versa. Antibodies useful in practicing the methods of the invention include (a) monoclonal antibodies, (b) purified polyclonal antibodies that specifically bind to the target polypeptide, (c) antibodies as described in (a)-(b) above that bind equivalent and highly homologous epitopes or phosphorylation sites in other non-human species (e.g. mouse, rat), and (d) fragments of (a)-(c) above that bind to the antigen (or more preferably the epitope) bound by the exemplary antibodies disclosed herein

By “antibody” or “antibodies” herein is meant all types of immunoglobulins, including IgG, IgM, IgA, IgD, and IgE. The antibodies may be monoclonal or polyclonal and may be of any species of origin, including (for example) mouse, rat, rabbit, horse, or human, or may be chimeric antibodies. See, e.g., M. Walker et al., Molec. Immunol. 26: 403-11 (1989); Morrision et al., Proc. Nat'l. Acad. Sci. 81: 6851 (1984); Neuberger et al., Nature 312: 604 (1984)). The antibodies may be recombinant monoclonal antibodies produced according to the methods disclosed in U.S. Pat. No. 4,474,893 (Reading) or U.S. Pat. No. 4,816,567 (Cabilly et al.) The antibodies may also be chemically constructed specific antibodies made according to the method disclosed in U.S. Pat. No. 4,676,980 (Segel et al.)

The invention is not limited to use of antibodies, but includes equivalent molecules, such as protein binding domains or nucleic acid aptamers, which bind, in a fusion-protein or truncated-protein specific manner, to essentially the same epitope to which an MYD88 L265P-specific antibody useful in the methods of the invention binds. See, e.g., Neuberger et al., Nature 312: 604 (1984). Such equivalent non-antibody reagents may be suitably employed in the methods of the invention further described below.

Polyclonal antibodies useful in practicing the methods of the invention may be produced according to standard techniques by immunizing a suitable animal (e.g., rabbit, goat, etc.) with an antigen encompassing a desired fusion-protein specific epitope (e.g. the fusion junction of an Rspo fusion protein described herein), collecting immune serum from the animal, and separating the polyclonal antibodies from the immune serum, and purifying polyclonal antibodies having the desired specificity, in accordance with known procedures. The antigen may be a synthetic peptide antigen comprising the desired epitopic sequence, selected and constructed in accordance with well-known techniques. See, e.g., ANTIBODIES: A LABORATORY MANUAL, Chapter 5, p. 75-76, Harlow & Lane Eds., Cold Spring Harbor Laboratory (1988); Czernik, Methods In Enzymology, 201: 264-283 (1991); Merrifield, J. Am. Chem. Soc. 85: 21-49 (1962)). Polyclonal antibodies produced as described herein may be screened and isolated as further described below.

Monoclonal antibodies may also be beneficially employed in the methods of the invention, and may be produced in hybridoma cell lines according to the well-known technique of Kohler and Milstein. Nature 265: 495-97 (1975); Kohler and Milstein, Eur. J. Immunol. 6: 511 (1976); see also, CURRENT PROTOCOLS IN MOLECULAR BIOLOGY, Ausubel et al. Eds. (1989). Monoclonal antibodies so produced are highly specific, and improve the selectivity and specificity of assay methods provided by the invention. For example, a solution containing the appropriate antigen (e.g. a synthetic peptide comprising the fusion junction of Rspo-PTPRK or Rspo-EIF3E fusion polypeptide) may be injected into a mouse and, after a sufficient time (in keeping with conventional techniques), the mouse sacrificed and spleen cells obtained. The spleen cells are then immortalized by fusing them with myeloma cells, typically in the presence of polyethylene glycol, to produce hybridoma cells. Rabbit fusion hybridomas, for example, may be produced as described in U.S. Pat. No. 5,675,063, K. Knight, Issued Oct. 7, 1997. The hybridoma cells are then grown in a suitable selection media, such as hypoxanthine-aminopterin-thymidine (HAT), and the supernatant screened for monoclonal antibodies having the desired specificity, as described below. The secreted antibody may be recovered from tissue culture supernatant by conventional methods such as precipitation, ion exchange or affinity chromatography, or the like.

Monoclonal Fab fragments may also be produced in Escherichia coli by recombinant techniques known to those skilled in the art. See, e.g., W. Huse, Science 246: 1275-81 (1989); Mullinax et al., Proc. Nat'l Acad. Sci. 87: 8095 (1990). If monoclonal antibodies of one isotype are preferred for a particular application, particular isotypes can be prepared directly, by selecting from the initial fusion, or prepared secondarily, from a parental hybridoma secreting a monoclonal antibody of different isotype by using the sib selection technique to isolate class-switch variants (Steplewski, et al., Proc. Nat'l. Acad. Sci., 82: 8653 (1985); Spira et al., J. Immunol. Methods, 74: 307 (1984)). The antigen combining site of the monoclonal antibody can be cloned by PCR and single-chain antibodies produced as phage-displayed recombinant antibodies or soluble antibodies in E. coli (see, e.g., ANTIBODY ENGINEERING PROTOCOLS, 1995, Humana Press, Sudhir Paul editor.)

Further still, U.S. Pat. No. 5,194,392, Geysen (1990) describes a general method of detecting or determining the sequence of monomers (amino acids or other compounds) that is a topological equivalent of the epitope (i.e., a “mimotope”) that is complementary to a particular paratope (antigen binding site) of an antibody of interest. More generally, this method involves detecting or determining a sequence of monomers that is a topographical equivalent of a ligand that is complementary to the ligand binding site of a particular receptor of interest. Similarly, U.S. Pat. No. 5,480,971, Houghten et al. (1996) discloses linear C₁-C-alkyl peralkylated oligopeptides and sets and libraries of such peptides, as well as methods for using such oligopeptide sets and libraries for determining the sequence of a peralkylated oligopeptide that preferentially binds to an acceptor molecule of interest. Thus, non-peptide analogs of the epitope-bearing peptides of the invention also can be made routinely by these methods.

Antibodies useful in the methods of the invention, whether polyclonal or monoclonal, may be screened for epitope and fusion protein specificity according to standard techniques. See, e.g. Czernik et al., Methods in Enzymology, 201: 264-283 (1991). For example, the antibodies may be screened against a peptide library by ELISA to ensure specificity for both the desired antigen and, if desired, for reactivity only with, e.g. an MYD88 L265P polypeptide of the invention and not with wild-type Rspo or wild-type MYD88. The antibodies may also be tested by Western blotting against cell preparations containing target protein to confirm reactivity with the only the desired target and to ensure no appreciable binding to wild-type MYD88. The production, screening, and use of fusion protein-specific antibodies is known to those of skill in the art, and has been described. See, e.g., U.S. Patent Publication No. 20050214301, Wetzel et al., Sep. 29, 2005.

MYD88 L265P-specific antibodies useful in the methods of the invention may exhibit some limited cross-reactivity with similar fusion epitopes in other fusion proteins or with the epitopes in wild type MYD88. This is not unexpected as most antibodies exhibit some degree of cross-reactivity, and anti-peptide antibodies will often cross-react with epitopes having high homology or identity to the immunizing peptide. See, e.g., Czernik, supra. Cross-reactivity with other fusion proteins is readily characterized by Western blotting alongside markers of known molecular weight. Amino acid sequences of cross-reacting proteins may be examined to identify sites highly homologous or identical to the MYD88 L265P polypeptide sequence to which the antibody binds. Undesirable cross-reactivity can be removed by negative selection using antibody purification on peptide columns (e.g. selecting out antibodies that bind either wild type MYD88).

MYD88 L265P specific antibodies of the invention that are useful in practicing the methods disclosed herein are ideally specific for human fusion polypeptide, but are not limited only to binding the human species, per se. The invention includes the production and use of antibodies that also bind conserved and highly homologous or identical epitopes in other mammalian species (e.g. mouse, rat, monkey). Highly homologous or identical sequences in other species can readily be identified by standard sequence comparisons, such as using BLAST, with a human MYD88 L265P.

Antibodies employed in the methods of the invention may be further characterized by, and validated for, use in a particular assay format, for example flow cytometry (FC), immunohistochemistry (IHC), and/or Immunocytochemistry (ICC). Antibodies may also be advantageously conjugated to fluorescent dyes (e.g. Alexa488, PE), or labels such as quantum dots, for use in multi-parametric analyses along with other signal transduction (phospho-AKT, phospho-Erk 1/2) and/or cell marker (cytokeratin) antibodies.

### C. Detection of MYD88 L265P Polynucleotide

MYD88 L265P-specific reagents provided by the invention also include nucleic acid probes and primers suitable for detection of an MYD88 L265P polynucleotide. Specific use of such probes in assays such as polymerase chain reaction (PCR) amplification is described herein.

In some embodiments, the MYD88 L265P is detected by PCR, such as regular PCR, Real-time PCR (Q-PCR) or digital PCR (including droplet digital PCR). A pair of primers is used to amplify the fusion genes. The primers are designed based on the fusion gene sequence to be amplified. Preferably, one primer hybridizes to a first sequence of an Rspo gene and the second primer hybridizes to a second sequence of a fusion partner gene. PCR can be performed on either cDNA (as prepared from RNA using the biological sample) or genomic DNA, under conditions that can be optimized as known in the art.

It shall be understood that all of the methods (e.g., PCR) that detect MYD88 L265P polynucleotides of the invention may be combined with other methods that detect MYD88 L265P polynucleotides or MYD88 L265P polypeptides. For example, detection of a MYD88 L265P polynucleotide in the genetic material of a biological sample (e.g., in a circulating tumor cell) may be followed by Western blotting analysis or immuno-histochemistry (IHC) analysis of the proteins of the sample to determine if the MYD88 L265P polynucleotide was actually expressed as a MYD88 L265P polypeptide in the biological sample. Such Western blotting or IHC analyses may be performed using an antibody that specifically binds to the polypeptide encoded by the detected MYD88 L265P polynucleotide.

In some embodiments, the MYD88 L265P gene is detected by hybridization using microarray where a custom fusion gene microarray is used to detect MYD88 L265P from cancer specimens. The oligos are designed to enable combined measurements of chimeric transcript junctions with exon-wise measurements of individual fusion partners. See Skotheim, R I; Thomassen, G O; Eken, M; Lind, G E; Micci, F; Ribeiro, F R; Cerveira, N; Teixeira, M R et al. A universal assay for detection of oncogenic fusion transcripts by oligo microarray analysis. Molecular Cancer 8: 5. (2009).

In some embodiments, the MYD88 L265P mutation is detected by hybridization using branched DNA assay. In these embodiments a custom hybridization and signal amplification assay, such as the branched DNA assay (QuantiGene®), is used to detect Rspo fusion transcripts in lysis solutions from cancer specimens. The sequences of capture extender probes and the label extender probes are derived from the exon sequences of MYD88 L265P gene.

In some embodiments, the MYD88 L265P mutation is detected by sequencing, such as Sanger sequencing or Next-generation sequencing.

Sequencing by extending a sequencing primer or by extending an extension product can be carried out using a variety of methods. For example, sequencing can be carried out with a labeled reversible terminator or by ligation with a labeled oligonucleotide. Sequencing can be performed using any commercially available method, such as a reversible terminator based sequencing method that is commercially available from companies such as Illumina, Inc. (San Diego, Calif.), and Life Technologies (Ion Torrent).

In some embodiments, high-throughput sequencing is performed using Clonal Single Molecule Array (Solexa, Inc/Illumina, Inc.) or sequencing-by-synthesis (SBS) utilizing reversible terminator chemistry. These technologies are described in part in, e.g., U.S. Pat. Nos. 6,969,488; 6,897,023; 6,833,246; 6,787,308; and US Publication Application Nos. 20040106130; 20030064398; 20030022207; and Constans, A., The Scientist 2003, 17(13):36.

### IV. Pharmaceutical Formulations and Administration

The present invention further relates to a pharmaceutical composition or a a pharmaceutical formulation comprising a compound of the invention or a pharmaceutically acceptable salt thereof, and one or more pharmaceutically acceptable carriers.

By “pharmaceutical composition” herein is meant the composition comprising an therapeutically effective amount of the oligonucleotide of the invention with or without a pharmaceutically acceptable carrier. The pharmaceutical compositions can comprise one or more oligonucleotides of the invention. The composition includes but not limited to aqueous or saline solutions, particles, aerosols, pellets, granules, powders, tablets, coated tablets, (micro) capsules, suppositories, syrups, emulsions, suspensions, creams, drops and other pharmaceutical compositions suitable for use in a variety of drug delivery systems. The compositions may be administered parenterally, orally, rectally, intravaginally, intraperitoneally, topically (in a dosage form as powders, ointments, gels, drops or transdermal patch), bucally, or as an oral or nasal spray. In all cases, the composition must be sterile and stable under the conditions of manufacture and storage and preserved against the microbial contamination. Pharmaceutical compositions of this invention for parenteral injection comprise pharmaceutically-acceptable sterile aqueous or nonaqueous solutions, dispersions, suspensions or emulsions, as well as sterile powders for reconstitution into sterile injectable solutions or dispersions just prior to use. The oligonucleotide of the invention can be suspended in an aqueous carrier, for example, in an isotonic buffer solution at a pH of about 3.0 to about 8.0, preferably at a pH of about 3.5 to about 7.4, 3.5 to 6.0, or 3.5 to about 5.0. The buffer solution includes sodium citrate-citric acid and sodium phosphate-phosphoric acid, and sodium acetate-acetic acid buffers. For oral administration, the composition will be formulated with edible carriers to form powders tablets, pills, dragees, capsules, liquids, gels, syrups, slurries, suspensions and the like. For solid compositions, conventional non-toxic solid carriers can include pharmaceutical grades of mannitol, lactose, starch, or magnesium stearate. For buccal administration, the composition will be tablets or lozenges in conventional manner. For inhalation, the composition will be an aerosol spray from pressurized packs or a nebulizer or a dry powder and can be selected by one of skill in the art. In some cases, in order to prolong the effect of the oligonucleotide of the invention, the oligonucleotides of the invention are also suitably administered by sustained-release systems. The oligonucleotide of the invention can be used in a liquid suspension of crystalline or amorphous material with poor water solubility to slow the releasing of the oligonucleotide. Alternatively, delayed releasing of a parenterally administered drug form of the oligonucleotide is accomplished by dissolving or suspending the oligonucleotide in hydrophobic materials (such as an acceptable oil vehicle). Injectable depot forms are made by entrapping the oligonucleotide in liposomes or microemulsions or other biodegradable semi-permeable polymer matrices such as polylactide-polyglycolide, poly (orthoesters) and poly (anhydrides).

The compounds described herein including pharmaceutically acceptable carriers such as addition salts or hydrates thereof, can be delivered to a patient using a wide variety of routes or modes of administration.

As used herein, the term “pharmaceutically acceptable carrier/excipient” includes any and all solvents, dispersion media, coatings, surfactants, antioxidants, preservatives (e.g., antibacterial agents, antifungal agents), isotonic agents, absorption delaying agents, salts, drugs, drug stabilizers, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except in so far as any conventional carrier is incompatible with the active ingredient, its use in the therapeutic or pharmaceutical compositions is contemplated.

As used herein, the term “pharmaceutically acceptable salts” refers to salts that retain the biological effectiveness and properties of the compounds of this invention and, which are not biologically or otherwise undesirable. In many cases, the compounds of the present invention are capable of forming acid and/or base salts by virtue of the presence of amino and/or carboxyl groups or groups similar thereto (e.g., phenol or hydroxyamic acid). Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like; particularly preferred are the ammonium, potassium, sodium, calcium and magnesium salts. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. The pharmaceutically acceptable salts of the present invention can be synthesized from a parent compound, a basic or acidic moiety, by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are preferred, where practicable. Lists of additional suitable salts can be found, e.g., in Remington's Pharmaceutical Sciences, 20th ed., Mack Publishing Company, Easton, Pa., (1985), which is herein incorporated by reference.

For clinical use, the oligonucleotides of the invention can be administered alone or formulated in a pharmaceutical composition via any suitable route of administration that is effective to achieve the desired therapeutic result. The “route” of administering the oligonucleotide of the invention shall mean the enteral, parenteral and topical administration or inhalation. The enteral routes of administration of the oligonucleotide of the invention include oral, gastric, intestinal, and rectal. The parenteral route includes intravenous, intraperitoneal, intramuscular, intrathecal, subcutaneous, local injection, vaginal, topical, nasal, mucosal, and pulmonary administration. The topical route of administration of the oligonucleotide of the invention denotes the application of the oligonucleotide externally to the epidermis, to the buccal cavity and into the ear, eye and nose.

As used herein, the terms “administering” or “administration” are intended to encompass all means for directly and indirectly delivering a compound to its intended site of action.

The compounds described herein, or pharmaceutically acceptable salts and/or hydrates thereof, may be administered singly, in combination with other compounds of the invention, and/or in cocktails combined with other therapeutic agents. Of course, the choice of therapeutic agents that can be co-administered with the compounds of the invention will depend, in part, on the condition being treated.

For example, when administered to patients suffering from a disease state caused by an organism that relies on an autoinducer, the compounds of the invention can be administered in cocktails containing agents used to treat the pain, infection and other symptoms and side effects commonly associated with the disease. Such agents include, e.g., analgesics, antibiotics, etc.

When administered to a patient undergoing cancer treatment, the compounds may be administered in cocktails containing anti-cancer agents and/or supplementary potentiating agents. The compounds may also be administered in cocktails containing agents that treat the side-effects of radiation therapy, such as anti-emetics, radiation protectants, etc.

Supplementary potentiating agents that can be co-administered with the compounds of the invention include, e.g., tricyclic anti-depressant drugs (e.g., imipramine, desipramine, amitriptyline, clomipramine, trimipramine, doxepin, nortriptyline, protriptyline, amoxapine and maprotiline); non-tricyclic and anti-depressant drugs (e.g., sertraline, trazodone and citalopram); Ca+2 antagonists (e.g., verapamil, nifedipine, nitrendipine and caroverine); amphotericin; triparanol analogues (e.g., tamoxifen); antiarrhythmic drugs (e.g., quinidine); antihypertensive drugs (e.g., reserpine); thiol depleters (e.g., buthionine and sulfoximine); and calcium leucovorin.

The active compound(s) of the invention are administered per se or in the form of a pharmaceutical composition wherein the active compound(s) is in admixture with one or more pharmaceutically acceptable carriers, excipients or diluents. Pharmaceutical compositions for use in accordance with the present invention are typically formulated in a conventional manner using one or more physiologically acceptable carriers comprising excipients and auxiliaries, which facilitate processing of the active compounds into preparations which, can be used pharmaceutically. Proper formulation is dependent upon the route of administration chosen.

For transmucosal administration, penetrants appropriate to the barrier to be permeated are used in the formulation. Such penetrants are generally known in the art.

For oral administration, the compounds can be formulated readily by combining the active compound(s) with pharmaceutically acceptable carriers well known in the art. Such carriers enable the compounds of the invention to be formulated as tablets, pills, dragees, capsules, liquids, gels, syrups, slurries, and suspensions for oral ingestion by a patient to be treated. Pharmaceutical preparations for oral use can be obtained solid excipient, optionally grinding a resulting mixture, and processing the mixture of granules, after adding suitable auxiliaries, if desired to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxyniethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations, which can be used orally, include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Injection is a preferred method of administration for the compositions of the current invention. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents may be added, such as the cross-linked polyvinyl pyrrolidone, agar, or alginic acid or a salt thereof such as sodium alginate.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances, which increase the viscosity of the suspension, such as sodium carboxymethyl cellulose, sorbitol, or dextran. Optionally, the suspension may also contain suitable stabilizers or agents, which increase the solubility of the compounds to allow for the preparation of highly, concentrated solutions. For injection, the agents of the invention may be formulated in aqueous solutions, preferably in physiologically compatible buffers such as Hanks's solution, Ringer's solution, or physiological saline buffer.

Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation or transcutaneous delivery (e.g., subcutaneously or intramuscularly), intramuscular injection or a transdermal patch. Thus, for example, the compounds may be formulated with suitable polymeric or hydrophobic materials (e.g., as an emulsion in an acceptable oil) or ion exchange resins, or as sparingly soluble derivatives, for example, as a sparingly soluble salt.

The pharmaceutical compositions also may comprise suitable solid or gel phase carriers or excipients. Examples of such carriers or excipients include calcium carbonate, calcium phosate, various sugars, starches, cellulose derivatives, gelatin, and polymers such as polyethylene glycols.

A preferred pharmaceutical composition is a composition formulated for injection such as intravenous injection and includes about 0.01% to about 100% by weight of the compound of the present invention, based upon 100% weight of total pharmaceutical composition.

The drug-ligand conjugate may be an antibody-cytotoxin conjugate where the antibody has been selected to target a particular cancer.

In some embodiments, the pharmaceutical composition of the present invention further comprises an additional therapeutic agent.

In some embodiments, the additional therapeutic agent is an anticancer agent.

In some embodiments, the additional anticancer agent is selected from an antimetabolite, an inhibitor of topoisomerase I and II, an alkylating agent, a microtubule inhibitor, an antiandrogen agent, a GNRh modulator or mixtures thereof.

In some embodiments, the additional therapeutic agent is a chemotherapeutic agent.

By “chemotherapeutic agent” herein is meant a chemical compound useful in the treatment of cancer. Examples are but not limited to: Gemcitabine, Irinotecan, Doxorubicin, 5-Fluorouracil, Cytosine arabinoside (“Ara-C”), Cyclophosphamide, Thiotepa, Busulfan, Cytoxin, TAXOL, Methotrexate, Cisplatin, Melphalan, Vinblastine and Carboplatin.

In some embodiments, the second chemotherapeutic agent is selected from the group consisting of tamoxifen, raloxifene, anastrozole, exemestane, letrozole, imatanib, paclitaxel, cyclophosphamide, lovastatin, minosine, gemcitabine, cytarabine, 5-fluorouracil, methotrexate, docetaxel, goserelin, vincristine, vinblastine, nocodazole, teniposide etoposide, gemcitabine, epothilone, vinorelbine, camptothecin, daunorubicin, actinomycin D, mitoxantrone, acridine, doxorubicin, epirubicin, or idarubicin.

### Kits

In another aspect, the present invention provides kits containing one or more of the compounds or compositions of the invention and directions for using the compound or composition. In an exemplary embodiment, the invention provides a kit for conjugating a linker arm of the invention to another molecule. The kit includes the linker, and directions for attaching the linker to a particular functional group. The kit may also include one or more of a cytotoxic drug, a targeting agent, a detectable label, pharmaceutical salts or buffers. The kit may also include a container and optionally one or more vial, test tube, flask, bottle, or syringe. Other formats for kits will be apparent to those of skill in the art and are within the scope of the present invention.

### Medical Use

Accordingly, in another aspect, the present invention provides a method of inhibiting proliferation of an MYD88 L265P positive tumor/cancer call comprising admnisgtering to the tumor cell the compounds of the present invention. In some embodiments, the tumor can be metastasis or non-metastasis.

By “cancer” or “tumor” herein is meant the pathological condition in humans that is characterized by unregulated cell proliferation. Examples include but are not limited to: carcinoma, lymphoma, blastoma, and leukemia. More particular examples of cancers include but are not limited to: lung (small cell and non-small cell), breast, prostate, carcinoid, bladder, gastric, pancreatic, liver (hepatocellular), hepatoblastoma, colorectal, head and neck squamous cell carcinoma, esophageal, ovarian, cervical, endometrial, mesothelioma, melanoma, sarcoma, osteosarcoma, liposarcoma, thyroid, desmoids, chronic myelocytic leukemia (AML), and chronic myelocytic leukemia (CML).

By “inhibiting” or “treating” or “treatment” herein is meant to reduction, therapeutic treatment and prophylactic or preventative treatment, wherein the objective is to reduce or prevent the aimed pathologic disorder or condition. In one example, following administering of a compound of the present invention, a cancer patient may experience a reduction in tumor size. “Treatment” or “treating” includes (1) inhibiting a disease in a subject experiencing or displaying the pathology or symptoms of the disease, (2) ameliorating a disease in a subject that is experiencing or displaying the pathology or symptoms of the disease, and/or (3) affecting any measurable decrease in a disease in a subject or patient that is experiencing or displaying the pathology or symptoms of the disease. To the extent a compound of the present invention may prevent growth and/or kill cancer cells, it may be cytostatic and/or cytotoxic.

By “therapeutically effective amount” herein is meant an amount of a compound provided herein effective to “treat” a disorder in a subject or mammal. In the case of cancer, the therapeutically effective amount of the drug may reduce the number of cancer cells, reduce the tumor size, inhibit cancer cell infiltration into peripheral organs, inhibit tumor metastasis, inhibit tumor growth to certain extent, and/or relieve one or more of the symptoms associated with the cancer to some extent.

In another aspect, the present invention provides a method of treating a MYD88 L265P positive tumor/cancer in a subject comprising administering to the subject a therapeutically effective amount of the compounds of the present invention. In some embodiments, the tumor or cancer can be at any stage, e.g., early or advanced, such as a stage I, II, III, IV or V tumor or cancer. In some embodiments, the tumor or cancer can be metastatic or non-metastatic. In the context of metastasis, the methods of the present invention can reduce or inhibit metastasis of a primary tumor or cancer to other sites, or the formation or establishment of metastatic tumors or cancers at other sites distal from the primary tumor or cancer therapy. Thus, the methods of the present invention include, among other things, 1) reducing or inhibiting growth, proliferation, mobility or invasiveness of tumor or cancer cells that potentially or do develop metastases (e.g., disseminated tumor cells, DTC); 2) reducing or inhibiting formation or establishment of metastases arising from a primary tumor or cancer to one or more other sites, locations or regions distinct from the primary tumor or cancer; 3) reducing or inhibiting growth or proliferation of a metastasis at one or more other sites, locations or regions distinct from the primary tumor or cancer after a metastasis has formed or has been established; and 4) reducing or inhibiting formation or establishment of additional metastasis after the metastasis has been formed or established.

In some embodiments, the tumor or cancer is solid or liquid cell mass. A “solid” tumor refers to cancer, neoplasia or metastasis that typically aggregates together and forms a mass. Specific non-limiting examples include breast, ovarian, uterine, cervical, stomach, lung, gastric, colon, bladder, glial, and endometrial tumors/cancers, etc. A “liquid tumor,” which refers to neoplasia that is dispersed or is diffuse in nature, as they do not typically form a solid mass. Particular examples include neoplasia of the reticuloendothelial or hematopoietic system, such as lymphomas, myelomas and leukemias. Non-limiting examples of leukemias include acute and chronic lymphoblastic, myeolblastic and multiple myeloma. Typically, such diseases arise from poorly differentiated acute leukemias, e.g., erythroblastic leukemia and acute megakaryoblastic leukemia. Specific myeloid disorders include, but are not limited to, acute promyeloid leukemia (APML), acute myelogenous leukemia (AML) and chronic myelogenous leukemia (CML). Lymphoid malignancies include, but are not limited to, acute lymphoblastic leukemia (ALL), which includes B-lineage ALL (B-ALL) and T-lineage ALL (T-ALL), chronic lymphocytic leukemia (CLL), prolymphocyte leukemia (PLL), hairy cell leukemia (HLL) and Waldenstroem's macroglobulinemia (WM). Specific malignant lymphomas include, non-Hodgkin lymphoma and variants, peripheral T cell lymphomas, adult T cell leukemia/lymphoma (ATL), cutaneous T-cell lymphoma (CTCL), large granular lymphocytic leukemia (LGF), Hodgkin's disease and Reed-Sternberg disease.

In some embodiments, the methods of the present invention can be practiced with other treatments or therapies (e.g., surgical resection, radiotherapy, ionizing or chemical radiation therapy, chemotherapy, immunotherapy, local or regional thermal (hyperthermia) therapy, or vaccination). Such other treatments or therapies can be administered prior to, substantially contemporaneously with (separately or in a mixture), or following administration of the compounds of the present invention.

In some embodiments, the methods of the present invention comprise administering a therapeutically effective amount of a compound of the present invention in combination with an additional therapeutic agent. In some embodiments, the additional therapeutic agent is an anticancer/antitumor agent. In some embodiments, the additional therapeutic agent is an antimetabolite, an inhibitor of topoisomerase I and II, an alkylating agent, a microtubule inhibitor, an antiandrogen agent, a GNRh modulator or mixtures thereof. In some embodiments, the additional therapeutic agent is selected from the group consisting of tamoxifen, raloxifene, anastrozole, exemestane, letrozole, imatanib, paclitaxel, cyclophosphamide, lovastatin, minosine, gemcitabine, cytarabine, 5-fluorouracil, methotrexate, docetaxel, goserelin, vincristine, vinblastine, nocodazole, teniposide etoposide, gemcitabine, epothilone, vinorelbine, camptothecin, daunorubicin, actinomycin D, mitoxantrone, acridine, doxorubicin, epirubicin, or idarubicin.

Administration “in combination with” one or more additional therapeutic agents includes simultaneous (concurrent) and consecutive administration in any order. As used herein, the term “pharmaceutical combination” refers to a product obtained from mixing or combining active ingredients, and includes both fixed and non-fixed combinations of the active ingredients. The term “fixed combination” means that the active ingredients, e.g. a compound of Formula (1) and a co-agent, are both administered to a patient simultaneously in the form of a single entity or dosage. The term “non-fixed combination” means that the active ingredients, e.g. a compound of Formula (1) and a co-agent, are both administered to a patient as separate entities either simultaneously, concurrently or sequentially with no specific time limits, wherein such administration provides therapeutically effective levels of the active ingredients in the body of the patient. The latter also applies to cocktail therapy, e.g. the administration of three or more active ingredients.

### Effective Dosages

Pharmaceutical compositions suitable for use with the present invention include compositions wherein the active ingredient is contained in a therapeutically effective amount, i.e., in an amount effective to achieve its intended purpose. The actual amount effective for a particular application will depend, inter alia, on the condition being treated. Determination of an effective amount is well within the capabilities of those skilled in the art, especially in light of the detailed disclosure herein.

The “therapeutically effective amount” of one of the oligonucleotides means a sufficient amount of the oligonucleotide used to achieve a desired result of treating or preventing an immune-mediated disorder, such as B-cell Lymphoma, in a subject. The oligonucleotides of the present invention may be employed in pure form or in pharmaceutically acceptable carriers. Alternatively, the oligonucleotides may be administered as pharmaceutical compositions. The “amount” in the invention shall refer to a dose. The dose can be determined by standard techniques well known to those skilled in the art and can vary depending the factors including, but not limited to the size or/and overall health of the subject or the severity of the disease. Introduction of the oligonucleotide of the invention can be carried out as a single treatment or over a series of treatments. Subject doses of the oligonucleotide of the invention for the administration range from about 1 μg to 100 mg per administration. However, doses for the treatment of immune-mediated disorder may be used in a range of 10 to 1,000 times higher than the doses described above. The more preferable doses can be adjusted to provide the optimum therapeutic effect by those skilled in the art, for example, by the attending physician within the scope of sound medical judgment. A therapeutically effective amount may be administered in one or more prophylactic or therapeutic administrations. When applied to an individual active ingredient, administered alone, the term refers to that ingredient alone. When applied to a combination, the term refers to combined amounts of the active ingredients that result in the therapeutic effect, whether administered in combination, serially or simultaneously.

For any compound described herein, the therapeutically effective amount can be initially determined from cell culture assays. Target plasma concentrations will be those concentrations of active compound(s) that are capable of inhibition cell growth or division. In preferred embodiments, the cellular activity is at least 25% inhibited. Target plasma concentrations of active compound(s) that are capable of inducing at least about 30%, 50%, 75%, or even 90% or higher inhibition of cellular activity are presently preferred. The percentage of inhibition of cellular activity in the patient can be monitored to assess the appropriateness of the plasma drug concentration achieved, and the dosage can be adjusted upwards or downwards to achieve the desired percentage of inhibition.

As is well known in the art, therapeutically effective amounts for use in humans can also be determined from animal models. For example, a dose for humans can be formulated to achieve a circulating concentration that has been found to be effective in animals. The dosage in humans can be adjusted by monitoring cellular inhibition and adjusting the dosage upwards or downwards, as described above.

A therapeutically effective dose can also be determined from human data for compounds which are known to exhibit similar pharmacological activities. The applied dose can be adjusted based on the relative bioavailability and potency of the administered compound as compared with the known compound.

Adjusting the dose to achieve maximal efficacy in humans based on the methods described above and other methods as are well-known in the art is well within the capabilities of the ordinarily skilled artisan.

In the case of local administration, the systemic circulating concentration of administered compound will not be of particular importance. In such instances, the compound is administered so as to achieve a concentration at the local area effective to achieve the intended result.

For use in the prophylaxis and/or treatment of diseases related to abnormal cellular proliferation, a circulating concentration of administered compound of about 0.001 μM to 20 μM is preferred, with about 0.01 μM to 5 μM being preferred.

Patient doses for oral administration of the compounds described herein, typically range from about 1 mg/day to about 10,000 mg/day, more typically from about 10 mg/day to about 1,000 mg/day, and most typically from about 50 mg/day to about 500 mg/day. Stated in terms of patient body weight, typical dosages range from about 0.01 to about 150 mg/kg/day, more typically from about 0.1 to about 15 mg/kg/day, and most typically from about 1 to about 10 mg/kg/day, for example 5 mg/kg/day or 3 mg/kg/day.

In at least some embodiments, patient doses that retard or inhibit tumor growth can be 1 μmol/kg/day or less. For example, the patient doses can be 0.9, 0.6, 0.5, 0.45, 0.3, 0.2, 0.15, or 0.1 μmol/kg/day or less (referring to moles of the drug). Preferably, the antibody with drug conjugates retards growth of the tumor when administered in the daily dosage amount over a period of at least five days.

For other modes of administration, dosage amount and interval can be adjusted individually to provide plasma levels of the administered compound effective for the particular clinical indication being treated. For example, in one embodiment, a compound according to the invention can be administered in relatively high concentrations multiple times per day. Alternatively, it may be more desirable to administer a compound of the invention at minimal effective concentrations and to use a less frequent administration regimen. This will provide a therapeutic regimen that is commensurate with the severity of the individual's disease.

Utilizing the teachings provided herein, an effective therapeutic treatment regimen can be planned which does not cause substantial toxicity and yet is entirely effective to treat the clinical symptoms demonstrated by the particular patient. This planning should involve the careful choice of active compound by considering factors such as compound potency, relative bioavailability, patient body weight, presence and severity of adverse side effects, preferred mode of administration and the toxicity profile of the selected agent.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

The oligonucleotides of the invention can be used alone, in combination with themselves, in a pharmaceutically acceptable carrier, in combination with one or more additional active ingredients. The administration of the oligonucleotide of the invention and additional active ingredients can be sequential or simultaneous. The active ingredients include non-steroidal anti-inflammatory agents, steroids, nonspecific immunosuppressive agent, biological response modifier, chemical compound, small molecule, nucleic acid molecule and TLR antagonists. The active ingredients also denote the agents that suppress the immune activation by antagonizing chemochines, by inducing the generation of regulatory T cells (CD4+CD25+ T cells), by inhibiting a complement, matrix metalloproteases and nitric oxide synthase, by blocking costimulatory factors and by inhibiting the signaling cascades in the immune cells. The non-steroidal anti-inflammatory agents include, but unlimited to, diclofenac, diflunisal, etodolac, flurbiprofen, ibuprofen, indomethacin, ketoprofen, ketorolac, nabumetone, naproxen, oxaprozin, piroxicam, sulindac, tohnetin, celecoxib and rofecoxib. The steroids include, but unlimited to, cortisone, dexamethasone, hydrocortisone, methylprednisolone, prednisolone, prednisone, and triamcinolone. A nonspecific immunosuppressive agent means the agent used to prevent the development of immune-mediated disorder. The nonspecific immunosuppressive agents include but not limited to cyclophosphamide, cyclosporine, methotrexate, steroids, FK506, tacrolimus, mycophenolic acid and sirolimus. The biological response modifier includes a recombinant interleukin-1-receptor antagonist (Kineret or anakima), a soluble p75 TNFα receptor-IgG1 fusion protein (etanercept or Enbrel), or a monoclonal antibody against TNFα (infliximab or RemicadeX). The agents also include Interferon beta-1α, interleukin-10 and TGFβ.

The oligonucleotides of the invention can be administered in/with a delivery vehicle or in a form linked with a vehicle. The vehicle includes, but not limited to, sterol (e.g., cholesterol), cochleates, emulsomes, ISCOMs; a lipid (e.g., a cationic lipid, anionic lipid), liposomes; ethylene glycol (PEG); live bacterial vectors (e.g., Salmonella, Escherichia coli, bacillus Calmette-Gurin, Shigella, Lactobacillus), live viral vectors (e.g., Vaccinia, adenovirus, Herpes simplex), virosomes, virus-like particles, microspheres, nucleic acid vaccines, polymers (e.g., carboxymethylcellulose, chitosan), polymer rings and a targeting agent that recognizes target cell by specific receptors.

Pegylation is the process of covalent attachment of poly (ethylene glycol) polymer chains to another molecule, normally a drug or therapeutic protein. Pegylation is routinely achieved by incubation of a reactive derivative of PEG with the target agent. The pegylated agent can “mask” the agent from the host's immune systems, increase the hydrodynamic size of the agent which prolongs its circulatory time. The oligonucleotides of the invention can be pegylated.

A pharmaceutically acceptable carrier denotes one or more solid or liquid filler, diluents or encapsulating substances that are suitable for administering the oligonucleotide of the invention to a subject. The carrier can be organic, inorganic, natural or synthetic. The carrier includes any and all solutions, diluents, solvents, dispersion media, liposome, emulsions, coatings, antibacterial and anti-fungal agents, isotonic and absorption delaying agents, and any other carrier suitable for administering the oligonucleotide of the invention and their use is well known in the art. The pharmaceutically acceptable carriers are selected depending on the particular mode of administration of the oligonucleotide. The parenteral formulations usually comprise injectable fluids that include pharmaceutically and physiologically acceptable fluids such as water, physiological saline, balanced salt solutions, aqueous dextrose, glycerol or the like as a vehicle. For solid compositions (e. g., powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

In some embodiments, the oligonucleotide is administered through the route of orall, enteral, parenteral, or topical administration, or inhalation.

### Combination Therapy

In another aspect, the present invention provides compostions and methods for combination therapy that the oligonucleotide is administered in combination with a chemotherapeutic agent, such as a Btk inhibitor, a PI3Kδ inhibitor, an IRAK inhibitor, an anti-CD20 monoclonal antibody, a SYK inhibitor, or a Bcl-2 inhibitor.

A “chemotherapeutic agent” is a chemical compound useful in the treatment of cancer. Examples are but not limited to: Gemcitabine, Irinotecan, Doxorubicin, 5-Fluorouracil, Cytosine arabinoside (“Ara-C”), Cyclophosphamide, Thiotepa, Busulfan, Cytoxin, TAXOL, Methotrexate, Cisplatin, Melphalan, Vinblastine and Carboplatin.

Administration “in combination with” one or more further therapeutic agents includes simultaneous (concurrent) and consecutive administration in any order. As used herein, the term “pharmaceutical combination” refers to a product obtained from mixing or combining active ingredients, and includes both fixed and non-fixed combinations of the active ingredients. The term “fixed combination” means that the active ingredients, e.g. an ODN of the present invention and a co-agent, are both administered to a patient simultaneously in the form of a single entity or dosage. The term “non-fixed combination” means that the active ingredients, e.g. an ODN of the present invention and a co-agent, are both administered to a patient as separate entities either simultaneously, concurrently or sequentially with no specific time limits, wherein such administration provides therapeutically effective levels of the active ingredients in the body of the patient. The latter also applies to cocktail therapy, e.g. the administration of three or more active ingredients. Such combination treatment may also include more than a single administration of the compound according to the invention and/or independently the other agent. The administration of the compound according to the invention and the other agent may be by the same or different routes.

B-cell receptor (BCR) is a transmembrane receptor protein located on the outer surface of B-cells. When a B-cell is activated by its first encounter with an antigen that binds to its receptor, the cell proliferates and differentiates to generate a population of antibody-secreting plasma B cells and memory B cells. BCR has two crucial functions upon interaction with Ag. One function is signal transduction, involving changes in receptor oligomerization. The second function is to mediate internalization for subsequent processing of Ag and presentation of peptides to helper T cells. BCR functions are required for normal antibody production, and defects in BCR signal transduction may lead to immunodeficency, autoimmunity and B-cell malignancy (Corcos D. et al. Blood 117 (26): 6991-8). B-Cell Receptor can follow through several signaling pathways, including the BTK, the PI3K and the IKK/NF-κB signaling pathways (Tomohiro K et al. Annual Review of Immunology 2008 28 (1): 21). B-cell receptor signalling is currently a therapeutic target in various lymphoid neoplasms.

Recently, BTK has emerged as a new anti-apoptotic molecular target for the treatment of B-lineage leukemias and lymphomas. BTK is predominantly expressed on B lymphocytes, lymphocyte precursors, and developing myeloid cells (Smith C I et al. J Immunol. 1994 557-565), and is essential in the chronic BCR activation (Davids M S et al. Leuk Lymphoma. 2012 2362-2370; Dal Porto J M et al. Mol Immunol. 2004 599-613). Upon activation of BCR, PI3K is activated, which in turn stimulates production of phosphatidylinositol-3,4,5 (PIP3). Once a sufficient amount of PIP3 is produced, BTK is recruited to the plasma membrane and then undergoes phosphorylation at the Y551 site by Src family kinases, especially LYN and FYN (Afar D E et al. Mol Cell Biol. 1996 3465-3471; Winer E S et al. Expert Opin Investig Drugs. 2012 355-361). Phosphorylated BTK activates phospholipase Cy2, leading to downstream activation of protein kinases (such as protein kinase C-beta) and, finally, activation of transcription factor NFkB. Therefore, IRAK and BTK appear to independently direct downstream NF-κB activation and combined use of IRAK and BTK inhibitors has been shown to lead to synergistic tumor cell killing in MYD88 (Yang G et al. 2012 JCO 8106). Ibrutinib (PCI-32765), a first-in-class BTK inhibitor, has demonstrated promising effectiveness in clinical development. Approximately 40% of patients with ABC DLBCL who had relapsed or were refractory to treatment responded to ibrutinib. But it does not seem to work in patients with the GCB subtype (Advani R H et al. J Clin Oncol. 2013 31:88-94), implying that the MYD88 mutation might play an important role in responding the treatment in ABC DLBCL paitents. In addition, there are many studies are to explore BTK inhibitor's synergistic efficacy in combination with other treatment. For example, a phase II study that showed ibrutinib combined with rituximab had profound activity (ORR=85%), and shortened the duration of re-distribution lymphocytosis in CLL patients with high-risk features (Burger J, Proc ASH Abst. 187).

As described above, PI3Ks play important roles in both TLRs and BCR signaling pathways. PI3Ks are lipid kinases that catalyze the generation of phosphatidylinositol-3, 4, 5-trisphosphate within the inner leaflet of the plasma membrane, creating binding sites for numerous intracellular Pleckstrin-homology domain-containing effectors, which trigger signal transduction cascades that control cell division, survival, metabolism, intracellular trafficking, differentiation, re-organization of the actin cytoskeleton, and cell migration (Vanhaesebroeck B et al. Rev Mol Cell Biol. 2010 329-341; Hawkins P T et al. Biochem Soc Trans. 2006 647-662). Therefore, inhibition of PI3K signaling can diminish cell proliferation, and in some circumstances, promote cell death. The class 1A PI3K enzymes are heterodimers comprised of a regulatory subunit (p85) and a catalytic subunit (p110) (Carpenter C L et al. J Biol. Chem. 1900 19704-11). p110a and p110β are ubiquitously expressed whereas p110γ and p110δ are predominantly expressed in leucocytes and at high levels in some cancer cell lines and human tissues of non-leukocyte origin such as breast cancer cells (Hu P et al. Mol Cell Biol. 1993 7677-88; Chantry D et al. Biol Chem. 1997 19236-41; Sawyer C et al. Cancer Res. 2003 1667-75). It was reported that p110β contributes to B cell development and maintenance, transformation, and proliferation (Hammer S B et al. J. Leukocyte Bio. 2010 1082-1095). The p110δ PI3K is over-activated in B cell malignancies because of alterations in BCR signaling and other signals provided by factors from tumor microenvironment (Pauls S D et al. Front Immunol. 2012 224-229). Higher levels of p110δ PI3K activity have been determined in cells from patients with chronic lymphocytic leukemia (CLL), multiple myeloma (MM) cell lines, and Hodgkin's lymphoma (HL) (Herman S E et al. Blood. 2010 2078-88; Ikeda H et al. Blood. 2010 1460-68; Meadows S A et al. Blood. 2012 1897-1900).

The critical role of p110δ in B cells led to the development of highly p110δ-specific inhibitors, including the initially developed idelalisib (CAL-101/GS-1101) for treatment of B-cell malignancies (Lannutti B J et al. Blood 2011 591-4). The activity of idelalisib and other p110δ-selective inhibitors have been studied in cell lines and patient cells from different B-cell malignancies including CLL and DLBCL (Ikeda H et al. Blood. 2010 1460-8; Hoellenriegel J et al. Blood. 2011 591-494). It was demonstrated that inhibition of p110δ by idelalisib reduces the B-CLL survival driven by B-cell molecules and furthermore acts by blocking cells to access protective niches inhibiting the environmental interactions that otherwise would promote B-cell survival and proliferation. Indeed, the phosphorylation of Akt in B-CLL and the plasma levels of CXCL13, CCL3, CCL4, and TNFα were found to be significantly reduced in patients treated with idelalisib, indicating that inhibition of p110δ disrupts the interactions of B-CLL from their protective microenvironment. Recently, idelalisib in combination with rituximab as a treatment for CLL has shown a highly statistically significant prolongation in the primary endpoint of progression-free survival (PFS) (ASH 55^{th} annual meeting 2013).

Rituximab is an anti-CD20 monoclonal antibody that was first approved by the FDA as an antineoplastic agent designed to treat B-cell malignancies. CD20 is a cell-surface marker specifically found on pre-B and mature B lymphocytes and is not found on other cell types and free in circulation (Maloney D G et al. Blood 1994 2457-2466). The binding of rituximab to cell surface CD20 located on the B lymphocytes results in destruction of the lymphocyte for the treatment of many lymphomas, leukemias, transplant rejection, and autoimmune disorders. Clinically, Rituximab alone was effective for the treatment of non-Hodgkin lymphomas (NHL), such as DLBCL (McLaughlin P et al. J Clin Oncol. 1998 2825-33; Sano T et al. International J Clin Oncol. 2007 59-62). Importantly, Rituximab, in combination with cyclophosphamide, doxorubicin, vincristine and prednisone (CHOP) chemotherapy, is superior to CHOP alone in the treatment of DLBCL and many other B-cell lymphomas (Hiddemann W et al. Blood 2005 3725-3732). Furthermore, adding ibrutinib to standard rituximab-CHOP chemotherapy for NHL resulted in an objective response rate of 100% (Younes A et al. J Clin Oncol. 2013 vol. 31 suppl. abs.8502). Other anti-CD20 treatments, including Veltuzumab, a pill version of humanized monoclonal antibody targeting CD20 receptors, are in clinical trials for the treatment of non-Hodgkin's lymphoma (NHL) and autoimmune diseases (Milani C et al. Curr Opin Mol Ther. 2009 200-207).

SYK is known to play an important role in transmitting maintenance signals and activation of the BCR. After BCR ligation by antigen, the Lyn PTK is activated and phosphorylates the immunoreceptor tyrosine-based activation (ITAM) motif in the cytoplasmic tail of receptor. Subsequent association of phosphorylated ITAM with SH2 domain of Syk leads to the activation of Syk by autophosphorylation. Syk autophosphorylation is necessary for the immune receptor-mediated activation of Syk, which is essential for activation of a cascade of signaling molecules including phosphatidylinositol 3-kinase, mitogen-activated protein kinases, Ras signaling pathways, phospholipase C-γ2 activation, and calcium mobilization (Tamir I et al. 1998 Oncogene 1353; Beitz L O et al. JBC 1999 32662-32666). The inhibition of Syk kinase represents a promising approach for the treatment of allergic and antibody-mediated autoimmune diseases, as well as for the treatment of lymphoma (Ulanova M et al. Expert Opin Ther Targets 2005 901-921; Friedberg J W et al. Blood. 2010 2578-2585). The most interesting Syk inhibitors in clinical trials are represented by R112, R406, R788 and R343. They are potent and selective ATP-competitive Syk kinase inhibitors developed at the Rigel Pharmaceuticals, Inc. Among them, R406 is able to inhibit tonic BCR signaling and induce apoptosis in some, but not all, primary DLBCLs (Chen L et al. Blood 2008 2230-2237). The orally active inhibitor R788 (fostamatinib) caused a dose-dependent improvement in disease severity in the treatment of rheumatoid arthritis and had significant clinical activity in NHL and CLL (Friedberg J W et al. Blood 2578-2585). Suppression of Syk kinase expression either by the antisense oligonucleotides (ASOs) or by the RNA interference (RNAi) is another approach to design of Syk inhibitors for the treatment of inflammatory diseases (Saitoh S et al. Immunity 2000 525-35). In addition, newer Syk inhibitors, such as GS-9973, are also in clinical development for treatment of NHL and CLL (Sharman J P et al. 2013 ASH Poster 1634).

Signaling by the aberrantly activated BCR plays a key role in the pathogenesis of certain types of B-cell tumors. As described above, BTK, and SYN all participate in BCR signaling pathways. Blocking BCR pro-survival pathway holds a great therapeutic potential in both NHL and CLL. Indeed, over the past several years, excitement has been building the clinical and therapeutic importance of BCR signaling in CLL and other B-cell lymphoproliferative disorders. Currently, the most active therapeutic regimens used for treatment of CLL are combinations of conventional chemotherapy together with the monoclonal anti-CD20 antibody rituximab, which has excellent overall response rates (Seiffert M et al. Blood 2011 3016-3024). However, efficacy is compromised in a subset of patients with loss of normal p53 activity due to deletion of chromosome 17p or somatic mutation of p53 (Zenz T et al. J Clin Oncol. 2010 4473-4479). In addition, these regimens are associated with significant morbidity as a result of myelo- and immune-suppression. Such toxicity often results in dose reduction or truncation of treatment and discourages use in elderly patients. Thus, new treatments that may hold benefit for patients with high-risk disease and that have a favorable therapeutic index are needed.

One strategy of combination therapy is to target both the TLR and BCR pathways simultaneously in synergy. This strategy is consistent with the observation that survival of ABC DLBCL cell lines requires a signal through both the TLR and the BCR as knocking down CD79 molecule of the BCR signaling compartment synergistically kills ABL-DLBCL cell lines with MYD88 knockdown (Ngo V N et al. Nature. 2011 115-119). Therefore synergistic killing of lymphoid neoplasms with L265P MYD88 using a combination of TLR9 antagnoist and BCR pathway inhibitors, such as ibrutinib (BTK inhibitor), Rituximab (anti-CD20 antibody), idelalisib (PI3K inhibitor) and R406 (SYK inhibitor) would be expected to improve the overall response rate and low toxicity in CLL patients.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### EXAMPLES

The present invention is further exemplified, but not limited, by the following and Examples that illustrate the preparation of the compounds of the invention.

The following oligonucleotides (ODNs) used in the example were synthesized in Takara Co. (Dalian, China). TLR9 stimulatory ODNs are:

CpG2395
(5′-tcgtcgttttcggcgcgcgccg-3′, SEQ ID NO.: 17),
CpG1826
(5′-tccatgacgttcctgacgtt-3′, SEQ ID NO.: 18),
CpG2216
(5′-gggggacgatcgtcgggggg-3′, SEQ ID NO.: 19).

Other ODNs used in the examples were:

(CCT)6
(5′-cctcctcctcctcctcct-3′, SEQ ID NO.: 15),
(CCT)7
(5′-cctcctcctcctcctcctcct-3′, SEQ ID NO.: 16),
(CCT)8
(5′-cctcctcctcctcctcctcctcct-3′, SEQ ID NO.: 20),
(CCT)8C
(5′-cctcctcctcctcctcctcctcctc-3′, SEQ ID NO.: 1),
(CCT)8CC
(5′-cctcctcctcctcctcctcctcctcc-3′, SEQ ID NO.: 2),
(CCT)9
(5′-cctcctcctcctcctcctcctcctcct-3′, SEQ ID NO.:
3),
(CCT)10
(5′-cctcctcctcctcctcctcctcctcctcct-3′, SEQ ID NO.:
6),
(CCT)10C
(5′-cctcctcctcctcctcctcctcctcctcctc-3′, SEQ ID
NO.: 7),
(CCT)10CC
(5′-cctcctcctcctcctcctcctcctcctcctcc-3′, SEQ ID
NO.: 8),
(CCT)11
(5′-cctcctcctcctcctcctcctcctcctcctcct-3′, SEQ ID
NO.: 9),
(CCT)11C
(5-cctcctcctcctcctcctcctcctcctcctcctc-3′, SEQ ID
NO.: 10),
(CCT)11CC
(5′-cctcctcctcctcctcctcctcctcctcctcctcc-3′, SEQ ID
NO.: 11),
(CCT)12
(5′-cctcctcctcctcctcctcctcctcctcctcctcct-3′, SEQ
ID NO.: 12),
(CCT)14
(5′-cctcctcctcctcctcctcctcctcctcctcctcctcctcct-3′,
SEQ ID NO.: 13)
and
(CCT)16
(5′-cctcctcctcctcctcctcctcctcctcctcctcctcctcctcctc
ct-3′, SEQ ID NO.: 14).

All reagents used to manipulate the oligonucleotides (ODNs) in the following examples arediluted in PBS and tested for endotoxin by Limulus amebocyte lysate assay (Accumedi Solutions Co., Ltd, Zhanjiang, China).

### Example 1

Diffuse Large B Cell Lymphoma (DLBCL) Cell Lines are Confirmed in the Presence of MYD88 L265P Mutant by Sequencing

Experimental Method

OCI-Ly3.3 bearing MYD88 L265P mutant and MYD88 wild type OCI-Ly19 were cultured in Iscove modified Dulbecco medium (IMDM, Hyclone, Logan, Utah, USA) complemented with 100 mg/mL of penicillin/streptomycin, and 10% and 20% fetal bovine serum. All cells were kept at 37° C. 5% CO2 in humid condition. Cell DNA was extracted from 3×10⁶ of OCI-Ly3.3 or OCI-Ly19 cells using genomic DNA kit (TransGen Biotech Co. Beijing, China), and polymerase chain reaction (PCR) was conducted using Tks Gflex DNA Polymerase (Takara Biotechnology, Dalian, China) with MYD 88 forward primer (5′-GTTGAAGACTGGGCTTGTCC-3′, SEQ ID NO.:51) and the reverse primer (5′-AGGAGGCAGGGCAGAAGTA-3′, SEQ ID NO.:52). The PCR products were extracted by gel extraction kit (Kangwei Biotechnology, Beijing, China) and cloned into pEasy-Blunt cloning kit (TransGen Biotech Co. Beijing, China). Two clones from each cell line were chosen for sequencing to detect MYD 88 L265P mutation by Genwiz Co. (Beijing, China).

Experimental Result

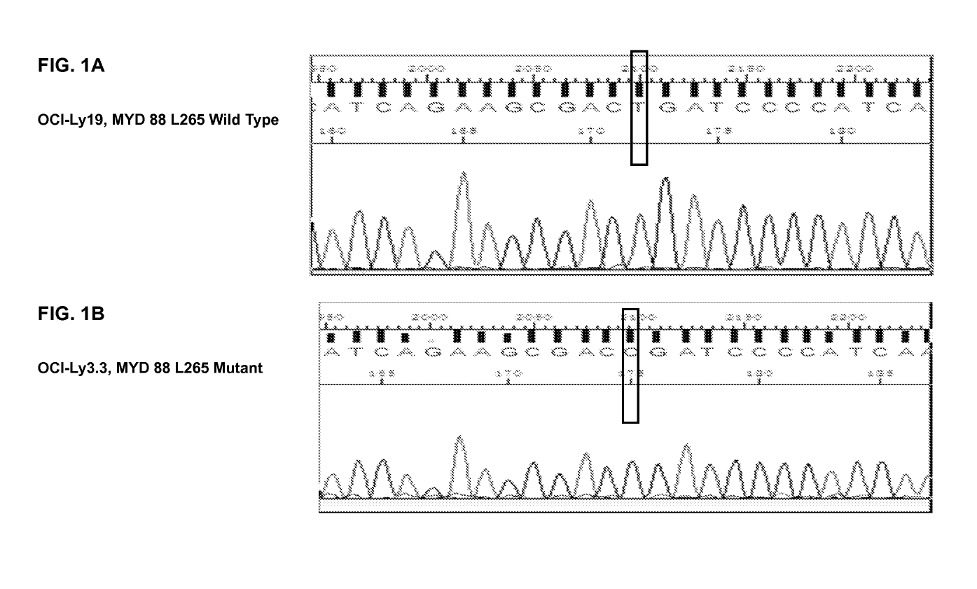

OCI-Ly3.3 was confirmed for the presence of MYD88 L265P mutant (FIG. 1, right) and OCI-Ly19 does not carry this mutant (FIG. 1, left). In consistent with previous report, OCI-Ly3.3 has homozygous MYD88 L265P mutant (FIG. 1, right).

### Example 2

Effect of TLR7/TLR9 Antagonists on ABC-DLBCL Cells with MYD88 L265P Mutant

Experimental Method

To observe the inhibitory effect of TLR7/9 antagonists on the proliferation of ABC-DLBCL cells, 5×10⁵/well of OCI-Ly3.3 and OCI-Ly19 cells in 96-well plate were cultured with TLR7/TLR9 antagonists as a dose range indicated below. The cell viability was measured by tetrazolium salt-based (WST-1), purchased from Beyotime Institute of Biotechnology (Jiangsu, China). The percentage of viable cells was calculated as a ratio of absorbance at 450 nm of treated to control cells.

To explore the cytokine secretion from DLBCL cells by TLR7/TLR9 antagonists, 2×10⁵/well of OCI-Ly19 or OCI-Ly 3.3 cells in 96-well plate were incubated with TLR7/9 antagonists, (CCT)₈, (CCT)₁₂ and (CCT)_{12-M}, corresponding the sequences ID of NOs. 20, 12 and 43 respectively, with the different concentrations indicated below. The supernatants were analyzed for cytokine IL-10 by cytometry beads assay after 36 hours of incubation.

Experimental Result

TLR7/9 antagonists were able to inhibit survival of OCI-Ly3.3 cells harboring MYD88 L265P mutation, but not wild type cells

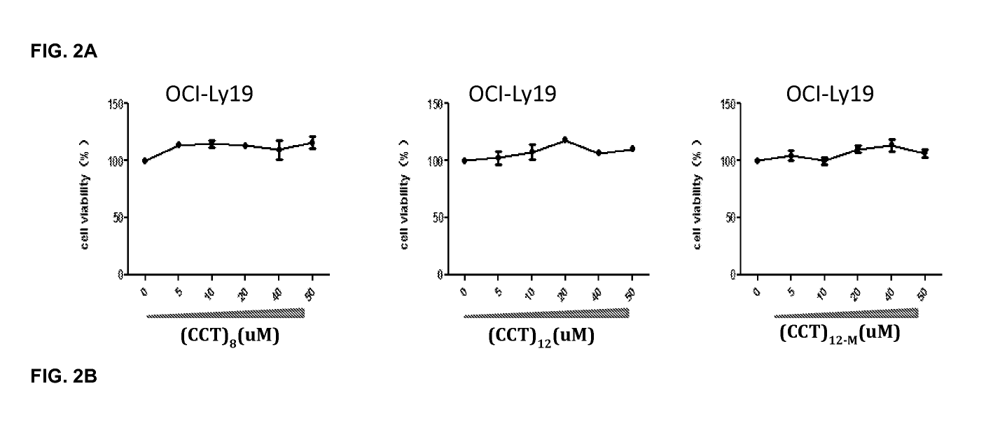

The maximum final concentration of vehicle did not induce any cytotoxicity on the cell lines tested (data not shown). 0.3 uM and 1 uM of (CCT)₈, (CCT)₁₂ and (CCT)_{12-M} led to an inhibition on OCI-Ly3.3 (FIG. 2B). but not OCI-Ly19 cells (FIG. 2A). Gating is a FACS dot-plot of CD19 (or CD3) and Side Scatter (SSC). Gate was set on the CD19 (or CD3) positive cells and the results represented percentage of CD19 or CD3 positive cells.

Decreased OCI-Ly3.3 Cell Viability is Associated with the Inhibition of Cytokines

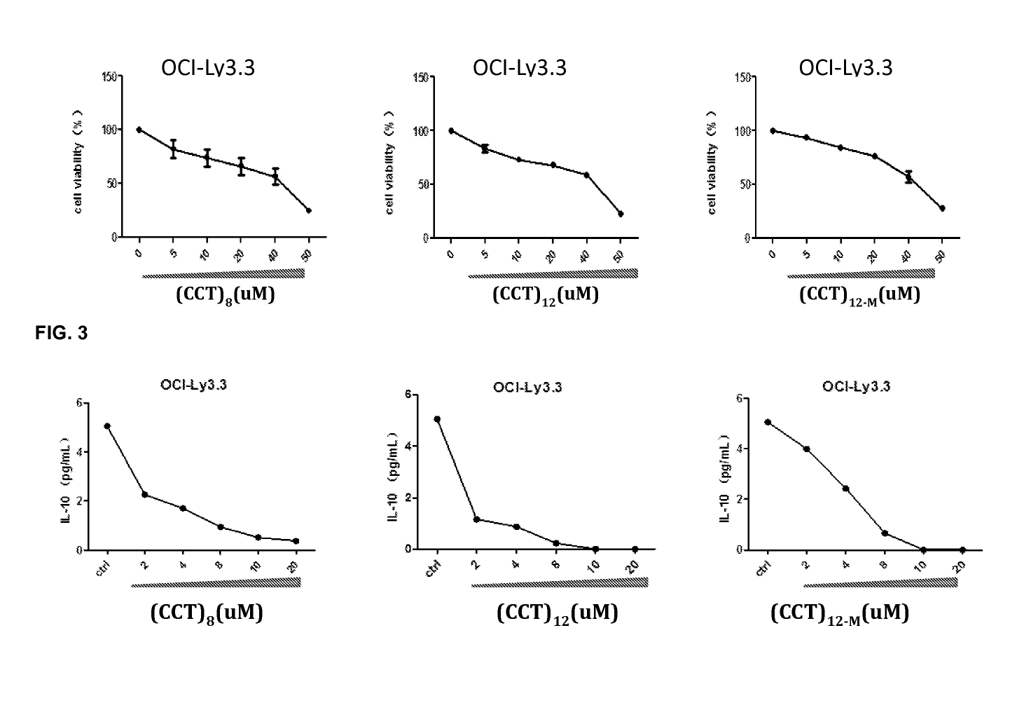

FIG. 3 showed that all the three TLR7/9 antagonists were able to inhibit IL-10 secretion in OCI-Ly3.3 cells. Data shown here are the mean values from three independent experiments. IL-10 cannot be detected in MYD88 L265 wild type OCI-Ly19 cells (data not shown).

1. A method for treating B-cell lymphoma in a subject that has been diagnosed as having a B-cell lymphoma characterized by a mutation in MYD88 and is in need of such treatment, comprising:
administering to said subject a pharmaceutical composition comprising a therapeutically effective amount of an oligonucleotide having a sequence of 5′-(CCT)ₙ-3′, wherein n is an integer from 2 to 50, and a pharmaceutically acceptable carrier.

2. The method of claim 1, wherein said oligonucleotide having a sequence of 5′-(CCT)ₙCm-3, n is an integer from 6 to 16, m is 0, 1, or 2.

3. The method of claim 1, wherein said B-cell lymphoma is selected from the group consisting of: Waldenström's macroglobulinemia (WM), activated B-cell-like (ABC) subtype of diffuse large B-cell lymphoma (DLBCL), and gastric mucosa-associated lymphoid tissue (MALT) lymphoma.

4. The method of claim 1, wherein said mutation in MYD88 comprises L265P, M232T, S243N, or T294P.

5. The method of claim 1, wherein said oligonucleotide comprises a sequence selected from the group consisting of:
(SEQ ID NO: 1)
5′-cctcctcctcctcctcctcctcctc-3′,
(SEQ ID NO: 2)
5′-cctcctcctcctcctcctcctcctcc-3′,
(SEQ ID NO: 3)
5′-cctcctcctcctcctcctcctcctcct-3′,
(SEQ ID NO: 4)
5′-cctcctcctcctcctcctcctcctcctc-3′,
(SEQ ID NO: 5)
5′-cctcctcctcctcctcctcctcctcctcc-3′,
(SEQ ID NO: 6)
5′-cctcctcctcctcctcctcctcctcctcct-3′,
(SEQ ID NO: 7)
5′-cctcctcctcctcctcctcctcctcctcctc-3′,
(SEQ ID NO: 8)
5′-cctcctcctcctcctcctcctcctcctcctcc-3′,
(SEQ ID NO: 9)
5′-cctcctcctcctcctcctcctcctcctcctcct-3′,
(SEQ ID NO: 10)
5′-cctcctcctcctcctcctcctcctcctcctcctc-3′,
(SEQ ID NO: 11)
5′-cctcctcctcctcctcctcctcctcctcctcctcc-3′,
(SEQ ID NO: 12)
5′-cctcctcctcctcctcctcctcctcctcctcctcct-3′,
(SEQ ID NO: 13)
5′-cctcctcctcctcctcctcctcctcctcctcctcctcctcct-3′,
(SEQ ID NO: 14)
5′-cctcctcctcctcctcctcctcctcctcctcctcctcctcctcctcc
t-3′,
(SEQ ID NO: 15)
5′-cctcctcctcctcctcct-3′,
and
(SEQ ID NO: 16)
5′-cctcctcctcctcctcctcct-3′.

6. The method of claim 1 wherein phosphate backbone of said oligonucleotide is unmodified.

7. The method of claim 1, wherein phosphate backbone of said oligonucleotide is partially or completely phosphorothioate-modified.

8. The method of claim 1, wherein said oligonucleotide comprises a chemical modification.

9. The method of claim 1, wherein said oligonucleotide further comprises one or more nucleotides to each end of said sequence of 5′-(CCT)ₙ-3′.

10. The method of claim 1, wherein said oligonucleotide is administered through the route of oral, enteral, parenteral, or topical administration, or inhalation.

11. The method of claim 1, wherein said oligonucleotide is administered in combination with a Btk inhibitor, a PI3Kδ inhibitor, an IRAK inhibitor, an anti-CD20 monoclonal antibody, a SYK inhibitor, or a Bcl-2 inhibitor.
